(19) United States
(12) Patent Application Publication
(10) Pub. No.: US 2005/0239809 A1
(43) Pub. Date: Oct. 27, 2005

(54) Methods for treating and preventing hypertension and hypertension-related disorders

(76) Inventors: Stephanie W. Watts, E. Lansing, MI (US); Carrie A. Northcott, E. Lansing, MI (US)

Correspondence Address: WILMER CUTLER PICKERING HALE AND DORR LLP, 60 STATE STREET, BOSTON, MA, 02109, US

(21) Appl. No.: 11/031,477

(22) Filed: Jan. 7, 2005

Related U.S. Application Data
(60) Provisional application No. 60/535,412, filed on Jan. 8, 2004.
(60) Provisional application No. 60/547,107, filed on Feb. 24, 2004.
(60) Provisional application No. 60/548,620, filed on Feb. 27, 2004.

(51) Int. Cl. A61K 31/52

(52) U.S. Cl. 514/263.21

(57) ABSTRACT

The present invention provides methods for treating hypertension and conditions associated with hypertension utilizing compounds that selectively inhibit PI-3-K p110δ activity.

### RELATED APPLICATIONS

This applications claims priority to U.S. provisional application 60/535,412, filed Jan. 8, 2004, and 60/547,107, filed Feb. 24, 2004, as well as 60/548,620, filed Feb. 27, 2004. These applications are incorporated herein by reference, in their entirety.

### FIELD OF THE INVENTION

The invention is in the field of the medical sciences. More specifically, the invention relates to methods and compounds for treating and preventing hypertension and secondary hypertension-related conditions by inhibiting vascular contraction using selective inhibitors of PI-3-Kδ (delta) activity.

### BACKGROUND OF THE INVENTION

High blood pressure or hypertension is a disease afflicting 20-30% of the world's adult population (Chobanian et al. (2003) JAMA 289: 2560-72). Hypertension presents with a myriad of altered cardiovascular endpoints, one of the most interesting being changes in arterial function and growth. Generally, arteries from animal models of hypertension and hypertensive humans are more sensitive to the ability of agonists to cause contraction, less responsive to agonists that cause relaxation, demonstrate spontaneous contractions in the absence of agonist and remodeling of the vessel through smooth muscle cell growth and hyperplasia (Lindop (1994) “The Effects of Hypertension on the Structure of Human Resistance Vessels” Swales, J. D. ed. Textbook of Hypertension. Oxford: Blackwell Scientific Publishers, 663-9; Lockette et al., (1986) Hypertension. 8: 61-6; Mulvany, (2002) News Physiol Sci. 17: 105-9; Safar et al. (1998) Hypertension 32: 156-61; Storm et al. (1990) Am. J. Hyperten. 3: 245S-48S; Thompson et al. (1987) Am. J. Cardiol. 59: 29A-34A.). The inappropriate growth observed in arteries from hypertensive subjects can be profound, and this dysregulation is not dissimilar to that occurring in cancer, another disease in which inappropriate cellular growth is present.

Spontaneous tone (non-agonist-induced contraction) is a phenomenon that is observed in both experimental and clinical forms of hypertension. Spontaneous tone has been observed in femoral arteries from renal hypertensive rats, DOCA-salt hypertensive rats, rats genetically predisposed to hypertension, essential hypertensive patients and women with preeclampsia Northcott, et al., supra; Hollenberg and Sandor, (1984) Hypertension 6: 579-585; Hollenberg, (1987) Am J Cardiol., 60(17): 571-601; Nilsson and Aalkjaer (2003) Mol Int.; 3(2): 79-89. Spontaneous tone development in the condition of hypertension leads to “spontaneous” narrowing of the arteries which can further increase/propagate the condition of hypertension by altering total peripheral resistance (TPR).

Two structurally unrelated pharmacological inhibitors of PI-3-kinase, LY294002 and wortmannin, inhibit aortic spontaneous tone observed in DOCA-salt rats in a concentration-dependent manner (Northcott, et al., (2002) Circ Res., 91: 360-369). Moreover, Class IA regulatory p85a subunit-associated PI-3-kinase activity and PI-3-kinase protein expression, specifically the p110δ subunit, is upregulated in aorta from DOCA-salt hypertensive rats compared to normotensive sham animals (Northcott, et al., (2002) Circ Res., 91: 360-369).

It is not apparent from these studies how different p110 isoforms play specific functional roles in these cells, or if any specific p110 isoform contributes to hypertension. Furthermore, the use of the nonspecific inhibitors of PI-3-K, wortmannin and LY294002, would not be practicable as a treatment option, since they would produce widespread deleterious effects on all PI-3-K mediated activities, including cellular growth and remodeling, as well as immune and cardiac function (see, e.g., Vlahos et al. (2003) Nat. Rev. Drug Discov. 2: 99-113). Accordingly, there exists a need to provide better forms of treatment that directly and specifically target the underlying molecular causes of hypertension and hypertension-related disorders.

### SUMMARY OF THE INVENTION

The invention is based, in part, upon the finding that the activity of a specific isoform of the p110 catalytic subunit, i.e., p100δ (p100delta), of phosphatidylinositol-3-kinase is central to the etiology of hypertension and hypertension-related disorders in mammals. Accordingly, the invention provides methods for treating hypertension using specific inhibitors of p100δ expression and/or activity, particularly the expression and/or activity of vascular p100δ.

In one aspect, the invention provides methods of ameliorating or preventing hypertension by administering to an individual an amount of a phosphoinositide 3-kinase delta (PI-3-Kδ) selective inhibitor effective to ameliorate or prevent hypertension and inhibit p110 delta (p110δ) activity. The invention further provides methods of ameliorating or preventing one or more conditions associated with hypertension, comprising administering to an individual an amount of a phosphoinositide 3-kinase delta (PI-3-Kδ) selective inhibitor effective to ameliorate or prevent the condition(s) associated with hypertension and inhibit vascular smooth muscle p110 delta (p110δ) activity. In one embodiment, methods contemplate inhibiting p110δ enzymatic activity directly, and in another embodiment, methods contemplate inhibiting p110δ enzymatic activity by inhibiting p110δ expression.

The term “selective PI-3-Kδ inhibitor” as used herein refers to a compound that inhibits the PI-3-Kδ isozyme more effectively than other isozymes of the PI-3-K family. A “selective PI-3-Kδ inhibitor” compound is understood to be more selective for PI-3-Kδ than compounds conventionally and generically designated PI-3-K inhibitors, e.g., wortmannin or LY294002. Concomitantly, wortmannin and LY294002 are deemed “nonselective PI-3-K inhibitors.”

Additionally, compounds of any type that selectively negatively regulate p110δ expression more effectively than other isozymes of the PI-3-K family, and that possess acceptable pharmacological properties can also be used as PI-3-Kδ selective inhibitors in the methods of the invention. Accordingly, in certain aspects, the invention provides for the use of antisense oligonucleotides which negatively regulate p110δ expression via hybridization to messenger RNA (mRNA) encoding p110δ, and to p110δ-targeting small interfering RNAs (siRNAs), which target the mRNA of p110δ for degradaion. In one embodiment, oligonucleotides that decrease p110δ expression and inhibit endothelial migration may be used in the methods of the invention. In additional embodiments, oligonucleotides that decrease p110δ expression and inhibit tubule formation may be used.

In another aspect, the invention provides a method of ameliorating or preventing hypertension or a condition associated with hypertension by administering to an individual an amount of a phosphoinositide 3-kinase delta (PI-3-Kδ) selective inhibitor effective to ameliorate or prevent hypertension, or a condition associated with hypertension, and inhibit vascular p110δ delta (p110δ). In certain useful embodiments, the p110δ activity is reduced, and in other embodiments, p110δ expression is reduced.

In certain embodiments of this aspect of the invention, the hypertension to be treated is essential hypertension. In other embodiments, the hypertension is secondary hypertension. In other embodiments, the condition associated with hypertension addressed is spontaneous tone, such as aortic spontaneous tone. In other embodiments, the condition is mesenteric resistance arterial spontaneous tone. In still other embodiments, the condition is enhanced arterial contraction, or enhanced total peripheral resistance.

In certain useful embodiments of the invention, the inhibitor is administered in a regimen which includes administering one or more additional therapeutic compounds such as ACE inhibitors, alpha-adrenoceptor agonists, alpha-adrenoceptor antagonists (alpha blockers), beta-adrenoceptor antagonists (beta blockers), angiotensin antagonists, atrial natriuretic factor, calcium channel antagonists, diuretics, dopamine receptor agonists, endopeptidase inhibitors, endothelin receptor antagonists, potassium channel agonists, renin inhibitors, serotonin antagonists, thromboxane antagonists and/or vasodilators.

In particularly useful embodiments of the invention the PI-3-Kδ selective inhibitor administered is a compound having formula (I) shown below, or a pharmaceutically acceptable salts or solvates thereof: 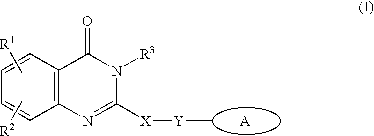
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen,
C₁₋₆alkyl, aryl, heteroaryl, halo, NHC(═O)C₁₋₃alkyleneN(R^{a})₂, NO₂, OR^{a}, CF₃,
OCF₃, N(R^{a})₂, CN, OC(═O)R^{a}, C(═O)OR^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkylene₂₋₄alkylene NR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneORₐ, C(═O)NR^{a}C₁₋₄alkylene C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})2, and OC₁₋₄alkyleneN(R^{a})₂, C¹⁻⁴alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C¹⁻⁴alkyleneOC¹⁻⁴alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃ alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C3₋₈cycloalkyl, aryl, and heteroaryl; and
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

In still further particularly useful embodiments of the invention, the PI-38δ selective inhibitor is one of the following chemical compounds: 2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2-chlorophenyl)-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7-dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9H-purin-6-yl-sulfanylmethyl)-3H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H=quinazolin-4-one; 3-cyclopropylmethyl-5-methyl-2-(9H-purin-6-ylsulfanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulfanylmethyl)-3-cyclopropylmethyl-5-methyl-3Hquinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclo-hexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro-4-oxo-3,4-dihydroquinazolin-2-ylmethyl ester; N-[3-(2-chlorophenyl)-5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5-methyl-3-o-tolyl-3H-quinazolin-4-one; 2-(4-amino-1,3;5-triazin-2-ylsulfanylmethyl)-5-methyl-3-o-tolyl-3H-quinazolin-4-one; 5-methyl-2-(7-methyl-7H-purin-6ylsulfanylmethyl)-3-o-tolyl-3H-quinazolin-4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-Diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3H-quinazolin-4-one; 3-isobutyl-5-methyl-2-(9H-purin-6-ylsulfanylmethyl)-3H-quinazolin-4-one; N-{2-[5-Methyl-4-oxo-2-(9H-purin-6-ylsulfanylmethyl)-4H-quinazolin-3-yl]-phenyl}-acetamide; 5-methyl-3-(E-2-methylcyclohexyl)-2-(9H-purin-6-ylsulfanylmethyl)-3H-quinazolin-4-one; 2-[5-methyl-4-oxo-2-(9H-purin-6-ylsulfanylmethyl)-4H-quinazolin-3-yl]-benzoic acid; 3-{2-[(2-dimethyl aminoethyl)methylamino]phenyl}-5-methyl-2-(9H-purin-6-ylsulfanylmethyl)-3H-quinazolin-4-one; 3-(2-chlorophenyl)-5-methoxy-2-(9H-purin-6-ylsulfanylmethyl)-3H-quinazolin-4-one; 3-(2-chlorophenyl)-5-(2-morpholin-4-yl-ethylamino)-2-(9H-purin-6-ylsulfanylmethyl)-3H-quinazolin-4-one; 3-benzyl-5-methoxy-2-(9H-purin-6-ylsulfanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[ ]-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; and 2-{2-(1-(6-aminopurin-9-ylmethyl)-5-methyl-4-oxo-4H-quinazolin-3-yl]-phenoxy}-acetamide, or any pharmaceutically acceptable salt or solvates thereof.

In a particularly useful embodiment, the invention provides the PI-38δ selective inhibitor is 2-(6-Amino-purin-9-ylmethyl)-5-methyl-3-o-tolyl-3H-quinazolin-4-one, having the structure 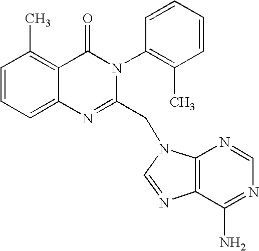
or any pharmaceutically acceptable salt or solvates thereof for use in the method of the invention.

In another particularly useful aspect, the invention provides a method of treating hypertension or a condition associated with hypertension by first identifying a subject with hypertension or a condition associated with hypertension; and then administering to the subject an amount of a phosphoinositide 3-kinase delta (PI3Kδ) selective inhibitor effective to treat the hypertension or the condition associated with hypertension, so that the hypertension, or a condition associated with hypertension, in the subject is treated.

In certain embodiments, the subject treated is a human. In other embodiments, the subject is a mammal. In still other useful embodiments, the subject treated is a rat or a mouse. In a particularly useful embodiment, the subject treated is a rat or mouse with genetically-based hypertension, such as an SHR rat. In other embodiments, the subject has a deoxycorticosterone acetate (DOCA)-salt induced hypertension.

In further embodiments of this aspect of the invention, the hypertension to be treated is essential hypertension. In other embodiments, the hypertension is secondary hypertension. In other embodiments, the condition associated with hypertension addressed is spontaneous tone, such as aortic spontaneous tone. In other embodiments, the condition is mesenteric resistance arterial spontaneous tone. In still other embodiments, the condition is enhanced arterial contraction, or enhanced total peripheral resistance.

In certain useful embodiments of the invention, the inhibitor is administered in a regimen which includes administering one or more additional therapeutic compounds such as ACE inhibitors, alpha-adrenoceptor agonists, alpha-adrenoceptor antagonists (alpha blockers), beta-adrenoceptor antagonists (beta blockers), angiotensin antagonists, atrial natriuretic factor, calcium channel antagonists, diuretics, dopamine receptor agonists, endopeptidase inhibitors, endothelin receptor antagonists, potassium channel agonists, renin inhibitors, serotonin antagonists, thromboxane antagonists and/or vasodilators.

In certain useful embodiments, the p110δ activity is reduced, and in other embodiments, p110δ expression is reduced.

In particularly useful embodiments of this aspect of the invention, the PI-3-Kδ selective inhibitor administered is a compound having formula (I) shown below, or a pharmaceutically acceptable salts or solvates thereof: 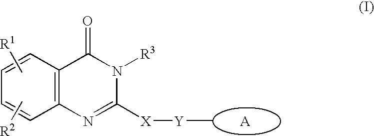
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen,
C₁₋₆alkyl, aryl, heteroaryl, halo, NHC(═O)C₁₋₃alkyleneN(R^{a})₂, NO₂, OR^{a}, CF₃,
OCF₃, N(R^{a})₂, CN, OC(═O)R^{a}, C(═O)OR^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C(═O)NR^{a}SO₂R^{a}, C₁₋₄alkyleneN(R^{a})2, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkylene₂₋₄alkylene NR^{a}C(═O)OR^{a}, NR^{a}C₁₋₄alkyleneN(R^{a})₂, NR^{a}C(═O)R^{a}, NR^{a}C(═O)N(R^{a})₂, N(SO2C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈gheterocycloalkyl, arylOC₁₋₃alkyleneN(R^{a})₂, arylOC(═O)R^{b}, NHC(═O)C₁₋₃alkyleneC₃₋₈gheterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneORₐ, C(═O)NR^{a}C₁₋₄alkylene C(═O)C₁₋₄alkyleneheteroaryl, C₁₋₄alkylenearyl optionally substituted with one or more of halo, SO₂N(R^{a})2, N(R^{a})2, C(═O)OR^{a}, NR^{a}SO₂CF₃, CN, NO₂, C(═O)R^{a}, OR^{a}, C₁₋₄alkyleneN(R^{a})2, and OC₁₋₄alkyleneN(R^{a})₂, C¹⁻⁴alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})2, C₁₋₄alkyleneOR^{a}, C¹⁻⁴alkyleneC(═O)R^{a}, C¹⁻⁴alkyleneOC¹⁻⁴alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃ alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

In still further particularly useful embodiments of the invention, the PI-38δ selective inhibitor is one of the following chemical compounds: 2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2chlorophenyl)-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-flhorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7-dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9H-purin-6-yl-sulfanylmethyl)-3H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H=quinazolin-4-one; 3-cyclopropylmethyl-5-methyl-2-(9H-purin-6-ylsulfanylmethyl)-3H-quinazolin-4-one; 2-(6-aminopurin-9-ylmethyl)-3-cyclopropylmethyl-5methyl-3H-quinazolin-4-one; 2-(2-amino-9H-purin-6-ylsulfanylmethyl)-3-cyclopropylmethyl-5-methyl-3Hquinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclo-hexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro-4-oxo-3,4-dihydroquinazolin-2-ylmethyl ester; N-[3-(2-chlorophenyl)-5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5r-methyl-3-o-tolyl-3H-quinazolin-4-one; 2-(4-amino-1,3;5-triazin-2-ylsulfanylmethyl)-5-methyl-3-o-tolyl-3H-quinazolin-4-one; 5-methyl-2-(7-methyl-7H-purin-6-ylsulfanylmethyl)-3-o-tolyl-3H-quinazolin-4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-Diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3H-quinazolin-4-one; 3-isobutyl-5-methyl-2-(9H-purin-6-ylsulfanylmethyl)-3H-quinazolin-4-one; N-(2-[5-Methyl-4-oxo-2-(9H-purin-6-ylsulfanylmethyl)-4H-quinazolin-3-yl]-phenyl}-acetamide; 5-methyl-3-(E-2-methylcyclohexyl)-2-(9H-purin-6-ylsulfanylmethyl)-3H-quinazolin-4-one; 2-[5-methyl-4-oxo-2-(9H-purin-6-ylsulfanylmethyl)-4H-quinazolin-3-yl]-benzoic acid; 3-{2-[(2-dimethyl aminoethyl)methylamino]phenyl}-5-methyl-2-(9H-purin-6-ylsulfanylmethyl)-3H-quinazolin-4-one; 3-(2-chlorophenyl)-5-methoxy-2-(9H-purin-6-ylsulfanylmethyl)-3H-quinazolin-4-one; 3-(2-chlorophenyl)-5-(2-morpholin-4-yl-ethylamino)-2-(9H-purin-6-ylsulfanylmethyl)-3H-quinazolin-4-one; 3-benzyl-5-methoxy-2-(9H-purin-6-ylsulfanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[ ]-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; and 2-{2-(1-(6-aminopurin-9-ylmethyl)-5-methyl-4-oxo-4H-quinazolin-3-yl]-phenoxy}-acetamide, or any pharmaceutically acceptable salt or solvates thereof.

In a particularly useful embodiment, the invention provides the PI-38δ selective inhibitor is 2-(6-Amino-purin-9-ylmethyl)-5-methyl-3-o-tolyl-3H-quinazolin-4-one, having the structure 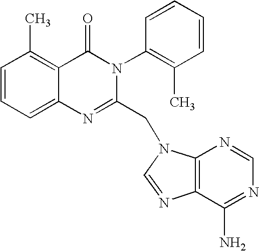
or any pharmaceutically acceptable salt or solvates thereof, for use in the method of the invention.

In another useful embodiment, the PI-3-Kδ selective inhibitor is an aptamer. In still further particularly useful embodiments, PI-3-Kδ selective inhibitor is a PI-3-Kδ targeted ribozyme, or a PI-3-Kδ targeted antisense oligonucleotide, or a PI-3-Kδ targeted siRNA.

### BRIEF DESCRIPTION OF THE FIGURES

FIG. 1A is a graphical representation of a spontaneous tone tracing showing LY294002-induced relaxation of endothelium-denuded mesenteric resistance arteries from DOCA-salt treated rats.

FIG. 1B is a quantitative graphical representation of relaxation induced by LY294002 compared to vehicle in DOCA-treated rats and in untreated control rats.

FIG. 2A shows a representation of a p85α Western blot, and a quantitative/graphical representation of the p85α Western blot, normalized to actin, in control and DOCA-treated rats.

FIG. 2B shows a representation of a p110δ Western blot, and a quantitative/graphical representation of the p110δ Western blot, normalized to actin, in control and DOCA-treated rats.

FIG. 2C shows representations of Akt/pAkt Western blots, and quantitative/graphical representation of the Akt/pAkt Western blots normalized to actin, in control and DOCA-treated rats.

FIG. 3A shows photographic representations of immunohistochemical images of rat thoracid aortae (RA) using an anti-p110δ antibody (right) or no primary antibody (left), and from DOCA-treated (bottom) or untreated (top) rats.

FIG. 3B shows a p110δ-associated PI-3-kinase assay (bottom), and a quantitative graphical representation of the results (top), of rat thoracid aortae from DOCA-treated (bottom) and control (Sham) rats.

FIG. 3C shows representations of p110δ, p110α, p110β and p110γ Western blots of p110δ antibody immunoprecipitates from aortic lysates of DOCA-salt induced hypertensive rats (DOCA) and control rats (Sham).

FIG. 4A is a graphical representation of a spontaneous tone tracing showing IC87114-induced relaxation of endothelium-denuded mesenteric resistance arteries from DOCA-salt treated rats, but not untreated rats.

FIG. 4B is a quantitative graphical representation of the results from FIG. 4A.

FIG. 4C is a quantitative graphical representation of the results of experiments showing a statistically significant decrease in spontaneous tone in aorta from DOCA-salt treated rats using nonspecific p110δ inhibitor LY294002 and the p110δ-specific inhibitor IC87114.

FIG. 5A is a graphical representation of spontaneous tone tracings from normal WKY and genetically hypertensive SHR rats.

FIG. 5B shows graphical representations of spontaneous tone tracings from normal WKY and genetically hypertensive SHR rats treated with PI-3 kinase inhibitor LY294002 or with a vehicle control.

FIG. 5C is a quantitative graphical representation of the magnitude of reduction in basal tone caused by LY294002 in WKY and SHR rat aortas.

FIG. 6 is a graphical representation of the results of experiments showing the effect of LY294002 on NE-induced contraction of aorta from normal WKY and hypertensive SHR rats.

FIG. 7A shows a representation of a p85 cc Western blot, and a quantitative/graphical representation of the p85 cc, Western blot, of rat aorta from normal WKY rats and genetically hypertensive SHR rats.

FIG. 7B shows a representation of a p110δ Western blot, and a quantitative/graphical representation of the p110δ Western blot, of rat aorta from normal WKY rats and genetically hypertensive SHR rats.

FIG. 7C shows a representation of a p110α Western blot, and a quantitative/graphical representation of the p110α Western blot, of rat aorta from normal WKY rats and genetically hypertensive SHR rats.

FIG. 7D shows a representation of a p110γ Western blot of rat aorta from normal WKY rats and genetically hypertensive SHR rats.

FIG. 8A shows representations of Akt and pAKT Western blots, and quantitative/graphical representations of Akt and pAKT Western blots, of rat aorta from normal WKY rats and genetically hypertensive SHR rats.

FIG. 8B shows representations of PTEN and pPTEN Western blots, and quantitative/graphical representations of PTEN and pPTEN Western blots, of rat aorta from normal WKY rats and genetically hypertensive SHR rats.

FIG. 9A is a schematic representation of the polypeptide sequence of a human PI-3-K p100δ subunit corresponding to GenBank Accession No. NP_{—}005017 (SEQ ID NO. 1).

FIG. 9B is a schematic representation of the nucleotide sequence of a human PI-3-K p100δ subunit corresponding to GenBank Accession No. NM_{—}005026 (SEQ ID NO. 2), wherein the initiation and termination codons of the vimentin protein open reading frame are underlined.

### DETAILED DESCRIPTION OF THE INVENTION

The patent and scientific literature referred to herein establishes knowledge that is available to those of skill in the art. The issued U.S. patents, allowed applications, published foreign applications, and references, including GenBank database sequences, that are cited herein are hereby incorporated by reference in their entirety to the same extent as if each was specifically and individually indicated to be incorporated by reference.

General

The invention is based, in part, upon the finding that the activity of a specific isoform of the p110δ catalytic subunit, i.e., p100δ (p100delta), of phosphatidylinositol-3-kinase is central to the etiology of hypertension and hypertension-related disorders in mammals. Accordingly, the invention provides methods for treating hypertension, and hypertension-related disorders, using specific inhibitors of p100δ expression and/or activity, particularly the expression and/or activity of vascular p100δ.

In general, methods of aspects of the invention contemplate treatment or prevention of primary hypertension, essential hypertension, or idiopathic hypertension arising from, but not limited to, genetic, environmental, dietary, rennin-affected, cell membrane defect, and insulin resistance factors; primary hypertension, essential hypertension, or idiopathic hypertension associated with, but not limited to, age, race, gender, smoking, alcohol consumption, serum cholesterol, glucose intolerance, and weight; systolic hypertension arising from decreased compliance of aorta (arteriosclerosis) and/or increased stroke volume related to, for example, aortic regurgitation, thyrotoxicosis, hyperkinetic heart syndrome, fever, arteriovenous fistula, and/or patent ductus arteriosus.

Methods of aspects of the invention further contemplate treatment or prevention of secondary hypertension, or systolic and diastolic hypertension, including renovascular hypertension associated with, for example, preeclampsia and eclampsia; renal vascular hypertension associated with, for example, chronic pyelonephritis, acute and chronic glomerulonephritis, polycystic renal disease, renovascular stenosis or renal infarction, severe renal disease such as, but not limited to, arteriolar nephrosclerosis and diabetic nephropathy, renin producing tumors such as, but not limited to, juxtaglomerular cell tumors and nephroblastomas; endocrine-related hypertension associated with oral contraceptive-induction, adenocortical hyperfunction associated with, but not limited to, Cushing's disease and syndrome, primary hyperaldosteronism, and/or congenital or hereditary adrenogenital syndromes (such as, for example, a 7α-hydroxylase defect and/or a 11β-hydroxylase defect), pheochromocytoma, myxedema, acromegaly, and hypercalcemia associated with, for example hyperparathyroidism, and more specifically, renal parenchymal damage, nephrolithiasis and/or nephrocalcinosis; neurogenic-related hypertension associated with, for example, pschyogenic conditions, diencephalic syndrome, familial dysautonomia (Riley-Day), polyneuritis associated with, for example acute porphyria and/or lead poisoning, increased intracranial pressure (acute) and/or spinal cord section (acute); hypertension associated with coarctation of aorta, increased intravascular volume (for example, excessive transfusion and/or polycythemia vera, polyarteritis nodosa, hypercalcemia, and/or medication-induction associated from use of, for example, glucocorticoids and/or cyclosporine; borderline hypertension, hypertensive crisis/emergency, intraoperative hypertension, perioperative hypertension, postoperative hypertension, labile hypertension, malignant hypertension, refractory hypertension, pulmonary hypertension, and/or white coat hypertension.

In providing methods of treatment of hypertension as described herein, an embodiment of the invention contemplates methods to treat secondary conditions associated with hypertension. With respect to the heart, embodiments of the invention provide methods to treat or prevent concentric left ventricular hypertrophy, ventricular signs of heart failure, angina pectoris, aortic regurgitation, ischemia, myocardial infarction and/or congestive heart failure. With respect to neurological condition, methods are provided to inhibit retinal changes, such as but not limited to focal spasm, narrowing of arterioles (arteriolosclerosis), appearance of, for example, hemorrhages, exudates and/or papilledema, scotomata, blurred vision and/or blindness; and/or central nervous system changes, including, but not limited to, occipital headaches, dizziness, vertigo, tinnitus, syncope, dim vision, vascular occlusion, hemorrhage, and/or encephalopathy. Methods are further provided for treatment or prevention of kidney disorders associated with hypertension including, but limited to, arteriosclerotic lesions of the afferent and efferent arterioles and glomerular capillary tufts, proteinuria, microscopic hematuria, renal failure, blood loss, epistaxis, emoptysis and/or metrorrhagia.

In further embodiments, the invention provides methods of treating spontaneous tone, comprising administering to an individual an amount of a phosphoinositide 3-kinase delta (PI-3-Kδ) selective inhibitor effective to inhibit or prevent spontaneous tone and inhibit p110 delta (p110δ). In one embodiment, the condition is aortic spontaneous tone. In another embodiment, the condition is mesenteric resistance arterial spontaneous tone. In still another embodiment, the condition is enhanced arterial contraction, and in yet another embodiment, the condition is enhanced total peripheral resistance.

In further embodiments, the invention provides methods wherein the phosphoinositide 3-kinase delta (PI-3-Kδ) selective inhibitor is administered in a regimen which includes administering one or more additional therapeutic compounds commonly utilized in hypertension treatment including, for example, diuretics, antiadrenergic agents, vasodilators, angiotensin-converting enzyme inhibitors, and/or calcium channel antagonists. Exemplary diuretics include, but are not limited to, thiazides (e.g., Hydrochlorothiazide), loop-acting diuretics (e.g., Furosemide) and/or potassium-sparing diuretics (e.g., Spironolactone, Triamterene, and/or Amiloride). Exemplary antiadrenergic agents include, but are not limited to, commercially-available Clonidine, Guanabenz, Guanfacine, Methyldopa, Trimethaphan, Guanethidine, Guanadrel, Phentolamine, Phenoxybenzamine, Prazosin, Terazosin, Doxazosin, Propanolol, Metaprolol, Nadolol, Atenolol, Timolol, Betaxolol, Carteolol, Pindolol, Labetalol, and/or Carvediol. Exemplary vasodilators include, for example, Hydralazine, Minoxidol, Diazaxide, and/or Nitroprusside. Exemplary angiotensin-converting enzyme inhibitors include, for example, Captopril, Benazepril, Enalapril, Enalaprilat, Fosinopril, Lisinopril, Quinapril, Ramipril and/or Trandolapril. Exemplary angiotensin receptor antagonists include, for example, Losartan, Valsartan and/or Irbesartan. Exemplary calcium channel antagonists include, for example, dihydropyridines such as Nifedipine XL, Amlodipine, Felodipine XL, Isradipine and/or Nicardipine, benzothiazepines such as Diltiazem and/or phehylalkylamines such as Verapamil.

Aspects of the invention contemplate methods wherein the phosphoinositide 3-kinase delta (PI-3-Kδ) selective inhibitor is administered in a regimen which includes administering one or more additional therapeutic compounds, beyond those disclosed but otherwise known in the art, including alpha-adrenoceptor agonists, alphaadrenoceptor antagonists (alpha blockers), beta-adrenoceptor antagonists (beta blockers), angiotensin antagonists, atrial natriuretic factor, dopamine receptor agonists, endopeptidase inhibitors, endothelin receptor, antagonists, potassium channel agonists, renin inhibitors, serotonin antagonists, thromboxane antagonists, and/or PDE5 inhibitors.

Methods according to embodiments of the invention include administering formulations comprising an inhibitor of the invention with a particular cytokine, lymphokine, other hematopoietic factor, thrombolytic or anti-thrombotic factor, or anti-inflammatory agent.

More specifically and without limitation, methods of aspects of the invention comprise administering an inhibitor with one or more of TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, G-CSF, Meg-CSF, GM-CSF, thrombopoietin, stem cell factor, and/or erythropoietin. Pharmaceutical compositions in accordance with the invention may also include other known angiopoietins, for example, Ang-1, Ang-2, Ang-4, Ang-Y, and/or the human angiopoietin-like polypeptide, and/or vascular endothelial growth factor (VEGF). Representative growth factors for use in pharmaceutical compositions of the invention include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein 15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor, nerve growth factor receptor, neurotrophin-3, neurotrophin-4, placenta growth factor, placenta growth factor 2, platelet derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

In another aspect, methods may include administering an inhibitor with one or more other agents which either enhance the activity of the inhibitor or compliment its activity or use in treatment. Such additional factors and/or agents may produce a synergistic effect with an inhibitor of the invention, or to minimize side effects.

Definitions

All technical and scientific terms used herein, unless otherwise defined below, are intended to have the same meaning as commonly understood by one of ordinary skill in the art; references to techniques employed herein are intended to refer to the techniques as commonly understood in the art, including variations on those techniques or substitutions of equivalent or later-developed techniques which would be apparent to one of skill in the art. In order to more clearly and concisely describe the subject matter which is the invention, the following definitions are provided for certain terms which are used in the specification and appended claims.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedents such as “about” or “at least about,” it will be understood that the particular value forms another embodiment.

As used herein, the term “aptamer” means any polynucleotide, or salt thereof, having selective binding affinity for a non-polynucleotide molecule (such as a protein) via non-covalent physical interactions. An aptamer is a polynucleotide that binds to a ligand in a manner analogous to the binding of an antibody to its epitope. Inhibitory aptamers of the invention are those that selectively inhibit p100δ activity.

As used herein, the term “alkyl” is defined as straight chained and branched hydrocarbon groups containing the indicated number of carbon atoms, typically methyl, ethyl, and straight chain and branched propyl and butyl groups. The hydrocarbon group can contain up to 16 carbon atoms, for example, one to eight carbon atoms. The term “alkyl” includes “bridged alkyl,” i.e., a C₆-C₁₆ bicyclic or polycyclic hydrocarbon group, for example, norboinyl, adamantyl, bicyclo[2.2.2]octyl, bicyclo[2.2.1]heptyl, bicyclo[3.2.1]octyl, or decahydronaphthyl. The term “cycloalkyl” is defined as a cyclic C₃-C₈ hydrocarbon group, e.g., cyclopropyl, cyclobutyl, cyclohexyl, and cyclopentyl.

The term “alkenyl” is defined identically as “alkyl,” except for containing a carbon-carbon double bond. “Cycloalkenyl” is defined similarly to cycloalkyl, except a carbon-carbon double bond is present in the ring.

The term “alkylene” is defined as an alkyl group having a substituent. For example, the term “C₁₋₃alkylenearyl” refers to an alkyl group containing one to three carbon atoms, and substituted with an aryl group.

The term “heteroC₁₋₃alkyl” is defined as a C₁₋₃alkyl group further containing a heteroatom selected from O, S, and NR^{a}. For example, —CH₂OCH₃ or —CH₂CH₂SCH₃. The term “arylheteroC₁₋₃alkyl” refers to an aryl group having a heteroC₁₋₃alkyl substituent.

The term “halo” or “halogen” is defined herein to include fluorine, bromine, chlorine, and iodine.

The term “aryl,” alone or in combination, is defined herein as a monocyclic or polycyclic aromatic group, e.g., phenyl or naphthyl. Unless otherwise indicated, an “aryl” group can be unsubstituted or substituted, for example, with one or more, and in particular one to three, halo, alkyl, phenyl, hydroxyalkyl, alkoxy, alkoxyalkyl, haloalkyl, nitro, and amino. Exemplary aryl groups include phenyl, naphthyl, biphenyl, tetrahydronaphthyl, chorophenyl, fluorophenyl, aminophenyl, methylphenyl, methoxyphenyl, trifluoromethylphenyl, nitrophenyl, carboxyphenyl, and the like. The terms “arylC₁₋₃alkyl” and “heteroarylC₁₋₃alkyl” are defined as an aryl or heteroaryl group having a C₁₋₃alkyl substituent.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, such as halo, alkyl, hydroxy, hydroxyalkyl, alkoxy, alkoxyalkyl, haloalkyl, nitro, and amino. Examples of heteroaryl groups include thienyl, furyl, pyridyl, oxazolyl, quinolyl, isoquinolyl, indolyl, triazolyl, isothiazolyl, isoxazolyl, imidizolyl, benzothiazolyl, pyrazinyl, pyrimidinyl, thiazolyl, and thiadiazolyl.

The term “Het” is defined as monocyclic, bicyclic, and tricyclic groups containing one or more heteroatoms selected from the group consisting of oxygen, nitrogen, and sulfur. A “Het” group also can contain an oxo group (═O) attached to the ring. Nonlimiting examples of Het groups include 1,3-dioxolane, 2-pyrazoline, pyrazolidine, pyrrolidine, piperazine, a pyrroline, 2H-pyran, 4H-pyran, morpholine, thiopholine, piperidine, 1,4-dithiane, and 1,4-dioxane.

The term “selective PI-3-Kδ inhibitor” as used herein refers to a compound that inhibits the PI-3-Kδ isozyme more effectively than other isozymes of the PI-3-K family. A “selective PI-3-Kδ inhibitor” compound is understood to be more selective for PI-3-Kδ than compounds conventionally and generically designated PI-3-K inhibitors, e.g., wortmannin or LY294002. Concomitantly, wortmannin and LY294002 are deemed “nonselective PI-3-K inhibitors.”

p110δ Proteins and Nucleic Acids

Phosphoinositide 3-kinase (PI-3-K) is a signaling enzyme that plays key roles in cellular growth, remodeling, apoptosis and is implicated in modulating vascular contraction (Wymann and Pirola, (1998) Biochem. Biophys. Acta., 1436: 127-150; Anderson et al. (1999) J. Biol. Chem., 274: 9907-9910; Rameh et al. (1999) J Biol. Chem., 274: 8347-8350; Cantrell (2001) J. Cell Sci., 114: 1439-1445; Coelho and Leevers (2000) J. Cell Sci.: 113: 2927-2934; Vanhaesebroeck et al., (2001) Ann. Rev. Biochem., 70: 535-602; Northcott, et al., (2002) Circ Res., 91: 360-369; Yang et al. (2001) Am. J. Physiol. Heart Circ. Physiol., 280: H2144-H2152; Komalavilas, et al., (2001) J. Appl Physiol., 91: 1819-1827). PI-3-kinase possesses both lipid and protein kinase activity, giving it the ability to be involved with a great number of signaling pathways. Cloning of the catalytic subunits of PI-3-kinase led to organizing the multigene family into three main classes based on their substrate specificity, sequence homology and regulation. Class I PI-3-kinases are the most extensively investigated class and contained two subunits, one of which plays primarily a regulatory/adaptor role (p85α, β, p55γ and p101) and the other that maintains the catalytic role of the enzyme (p110 α, β, δ, and γ) (Wymann and Pirola, (1998) Biochem. Biophys. Acta., 1436:127-150; Anderson et al. (1999) J. Biol. Chem., 274: 9907-9910; Rameh et al (1999) J. Biol. Chem., 274: 8347-8350; Cantrell, (2001) J. Cell Sci., 114: 1439-1445; Coelho and Leevers, (2000) J. Cell Sci.; 113: 2927-2934; Vanhaesebroeck et al. (2001) Ann. Rev. Biochem., 70: 535-602).

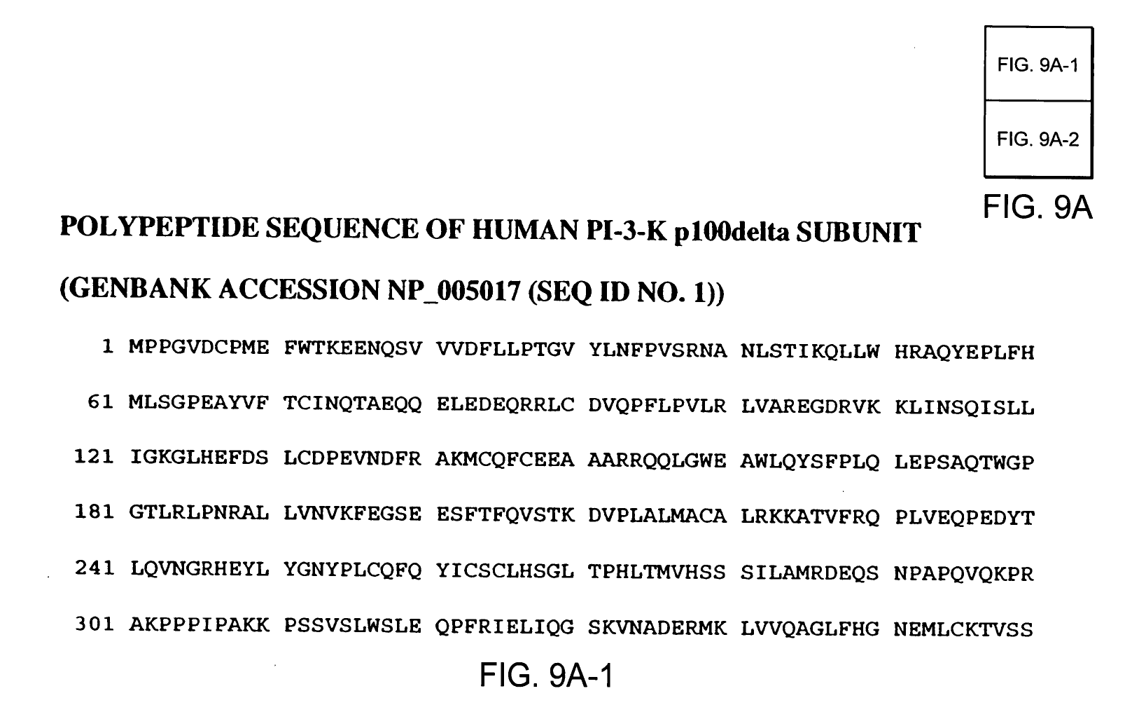

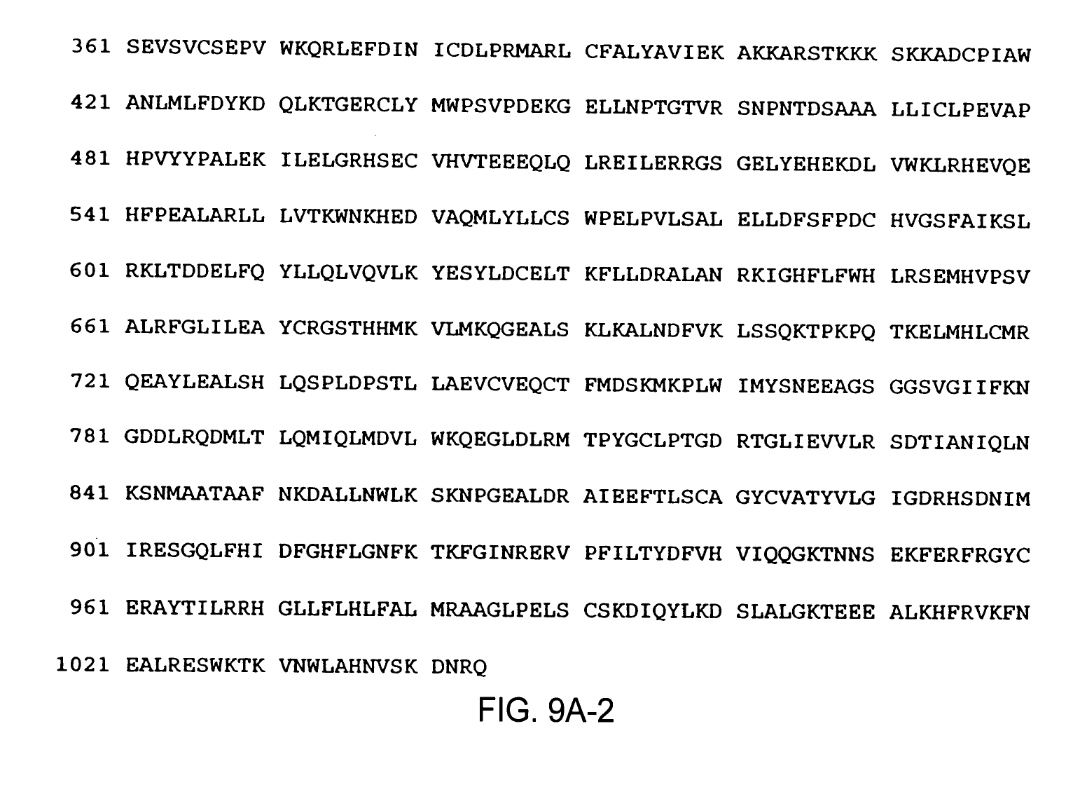

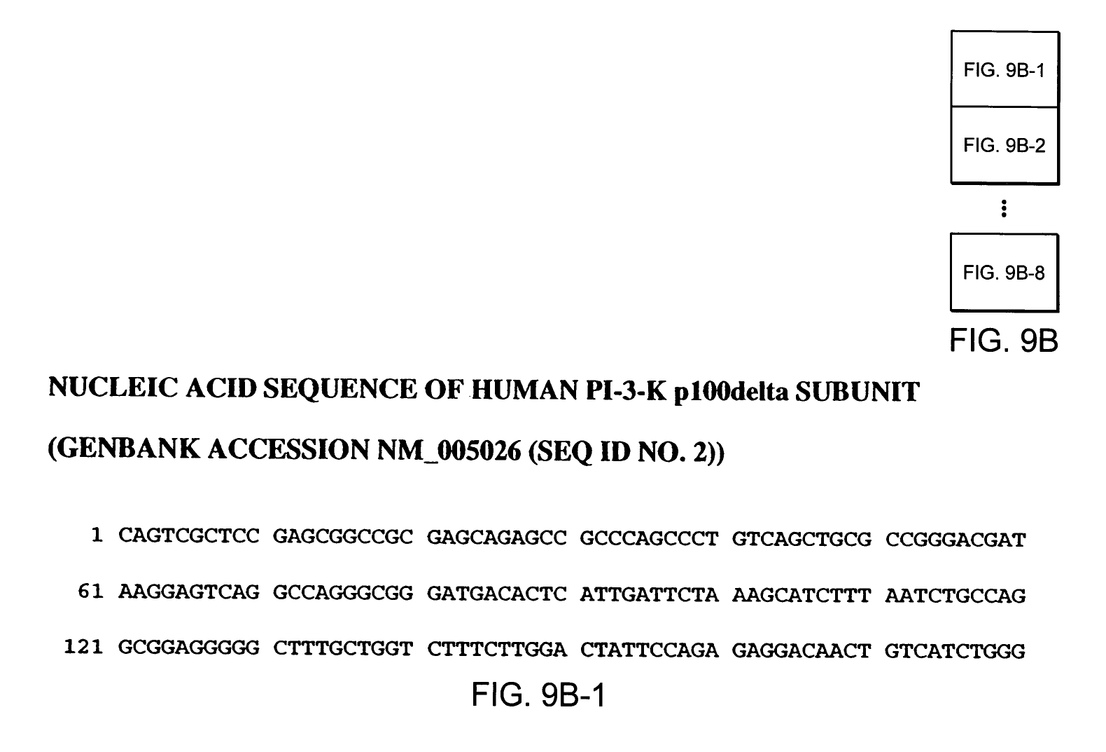

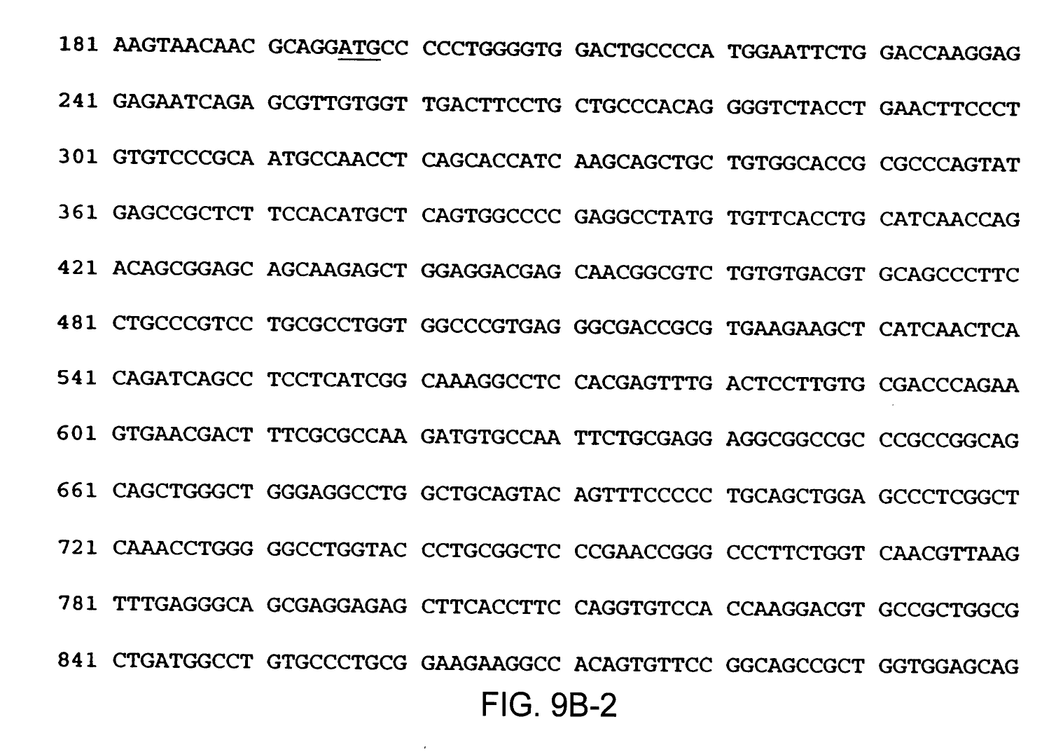

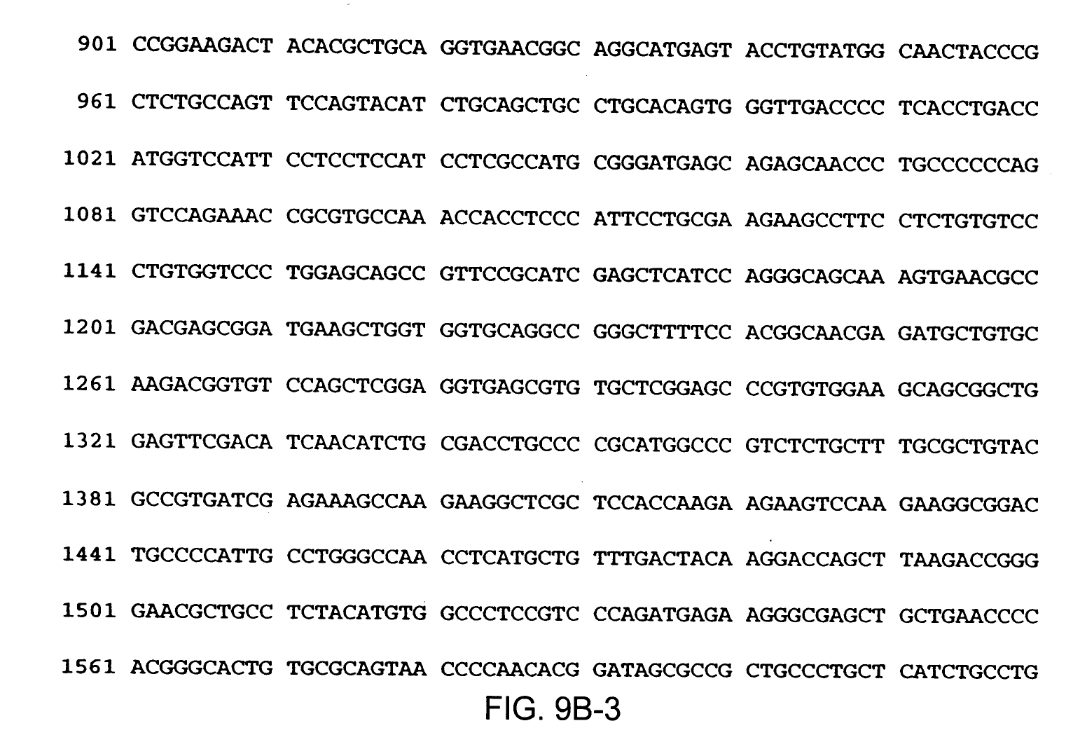

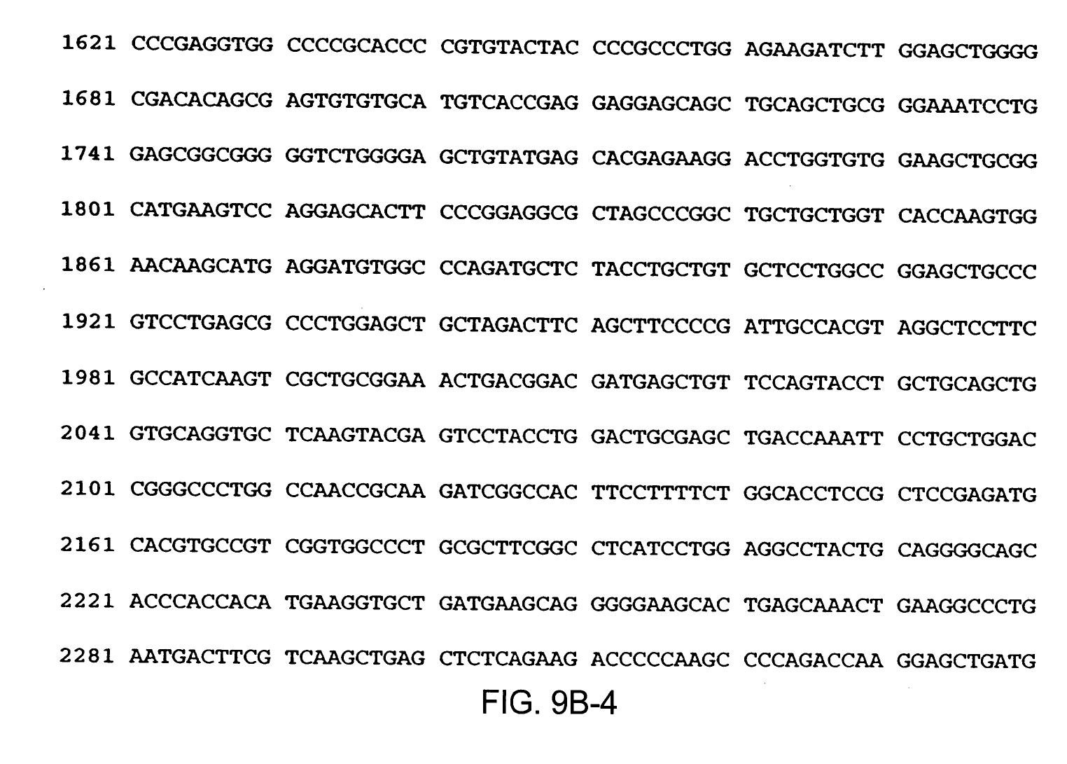

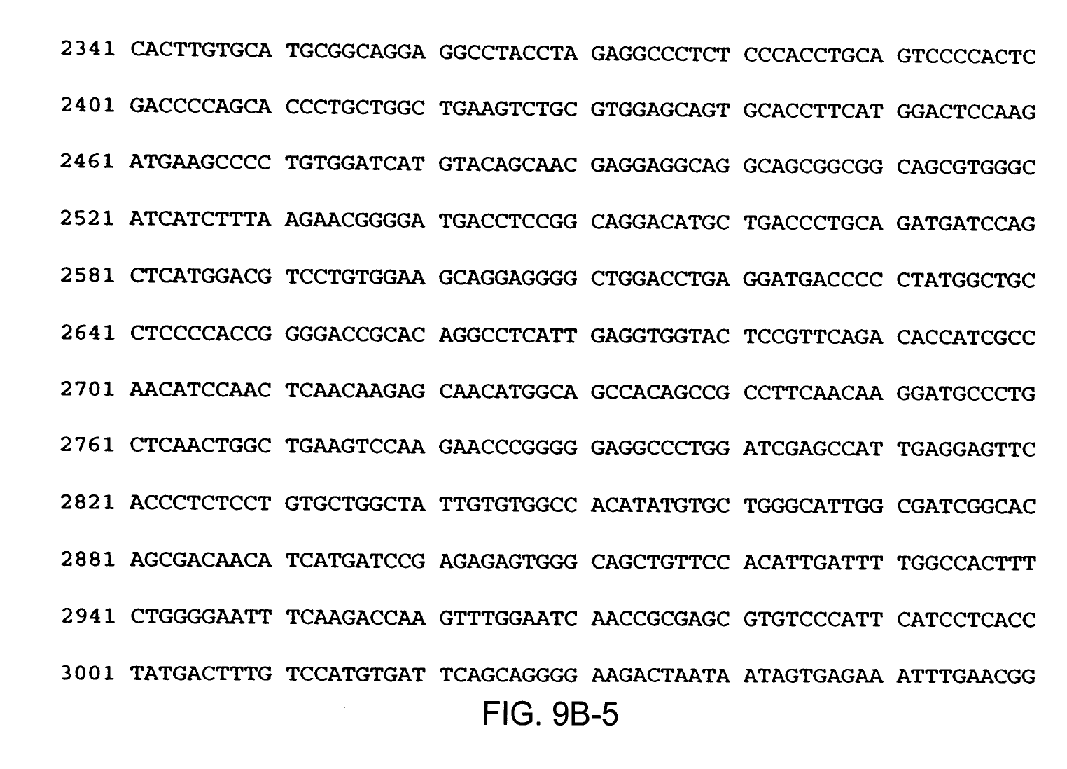

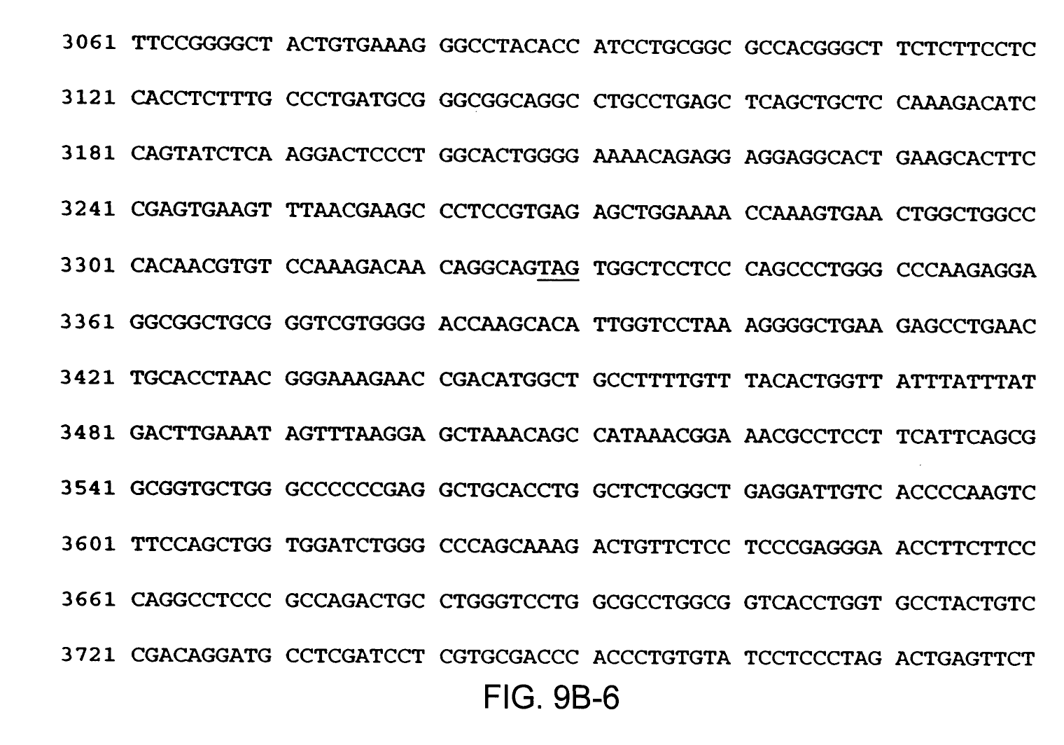

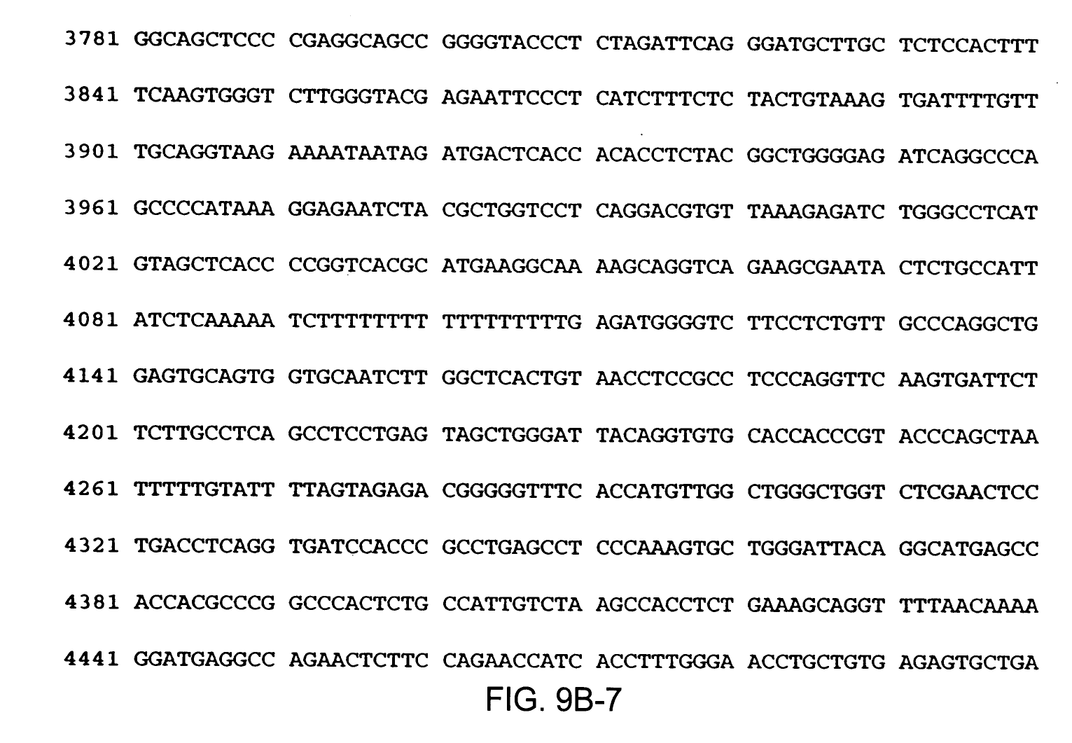

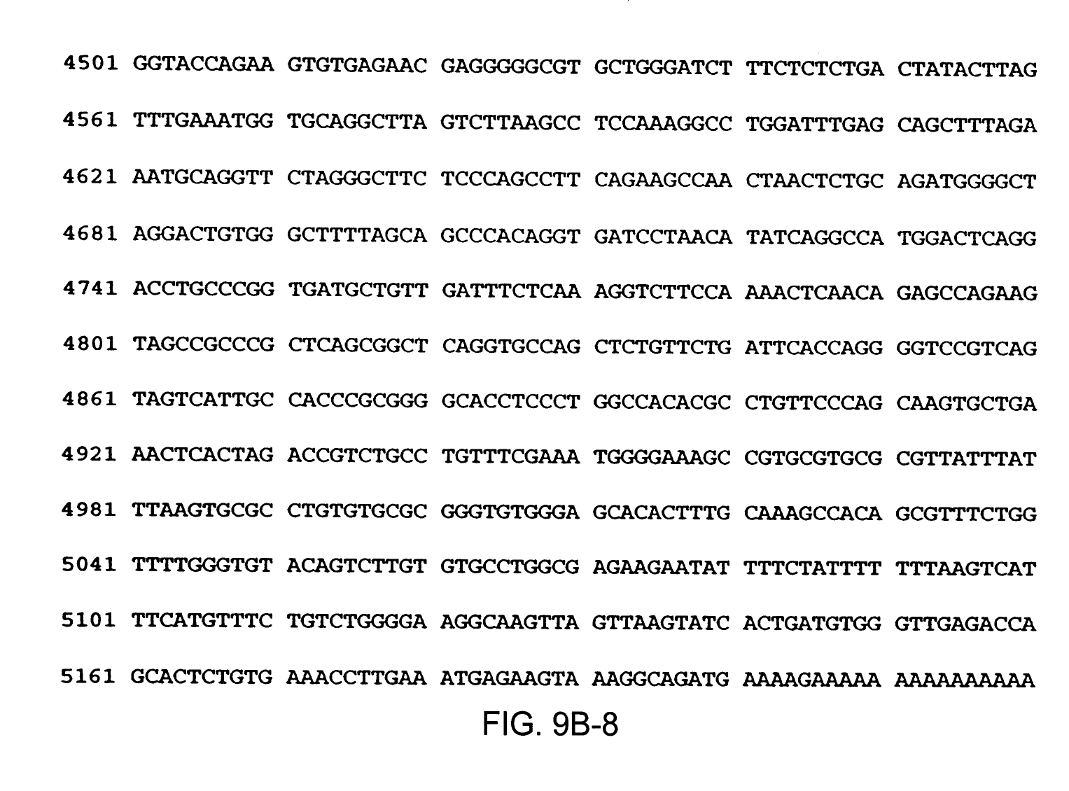

The nucleic acid and protein sequence of p100δ from various mammalian organisms are known in the art. For example, FIG. 9B shows the nucleic acid sequence of a human p100δ cDNA (corresponding to GenBank Accession NM_{—}005026), and FIG. 9A shows the corresponding human p100δ protein sequence (corresponding to GenBank Accession NP_{—}005017. Other p100δ nucleotide, and corresponding protein, sequences of the invention include: GenBank Accession Nos. U57843 and AAB53966; U86453 and AAC25677; and Y10055 and CAA71149. Nonlimiting exemplary p100δ nucleic acids and proteins for use in the invention are disclosed in U.S. Pat. Nos. 5,858,753, 5,882,910 and 5,985,589, the contents of which are hereby incorporated by reference herein, in their entireties.

Inhibitors of p110δ Activity

The invention includes the use of PI-3-Kδ selective chemical inhibitors for use in treating hypertension and hypertension related disorders. Nonlimiting, exemplary chemical inhibitors for use in the invention include those described in U.S. Pat. Nos. 6,518,277, 6,667,300, and 6,800,620, as well as PCT Publication WO 03/035075. Any selective inhibitor of PI-3-Kδ activity, including, but not limited to, small molecule inhibitors, peptide inhibitors non-peptide inhibitors, naturally occurring inhibitors, and synthetic inhibitors, may be used. For example, suitable PI-3-Kδ selective inhibitors have been described in to Sadhu et al. (see U.S. Pat. Nos. 6,518,277, 6,667,300, and 6,800,620, as well as PCT Publication WO 03/035075).

The relative efficacies of compounds as inhibitors of an enzyme activity (or other biological activity) can be established by determining the concentrations at which each compound inhibits the activity to a predefined extent and then comparing the results. Typically, the determination is the concentration that inhibits 50% of the activity in a biochemical assay, i.e., the 50% inhibitory concentration or “IC₅₀.” IC₅₀ determinations can be accomplished using conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀, etc.

Accordingly, a “selective PI-3-Kδ inhibitor” alternatively can be understood to refer to a compound that exhibits a 50% inhibitory concentration (IC₅₀) with respect to PI-3-Kδ that is at least 10-fold, in another aspect at least 20-fold, and in another aspect at least 30-fold, lower than the IC₅₀ value with respect to any or all of the other Class I PI-3-K family members. In an alternative embodiment of the invention, the term selective PI-3-Kδ inhibitor can be understood to refer to a compound that exhibits an IC₅₀ with respect to PI-3-Kδ that is at least 50-fold, in another aspect at least 100-fold, in an additional aspect at least 200-fold, and in yet another aspect at least 500-fold, lower than the IC₅₀ with respect to any or all of the other PI-3-K Class I family members. In yet a further embodiment, the term selective PI-3-Kδ inhibitor refers to an oligonucleotide that negatively regulates p110δ expression at least 10-fold, in another aspect at least 20-fold, and in a further aspect at least 30-fold, lower than any or all of the other Class I PI-3-K family catalytic subunits (i.e., p110α, p110β, and p110γ). A PI-3-Kδ selective inhibitor is administered to an individual in an amount such that the inhibitor retains its PI-3-Kδ selectivity, as described above.

Methods of aspects of the invention contemplate use of a PI-3-Kδ selective inhibitor compound having formula (1) or pharmaceutically acceptable salts and solvates thereof: 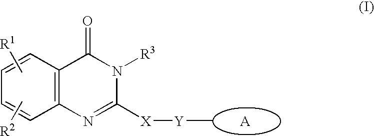
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF³, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(Ra)2, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkyleneN(R^{a})₂, C(═O)NR^{a}C₁₋₄alkyleneOR^{a}, C(═O)NR^{a}C₁₋₄alkyleneHet, OC₂₋₄ alkyleneN(R^{a})₂, C₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})2, NHC(═O)C₁₋₃alkylenearyl, C₃₋₈cycloalkyl, C₃₋₈gheterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkylene C(═O)OR^{b}, C(═O)OR^{b}, C(═O)C₁₋₄alkyleneHet, and NHC(═O)haloC₁₋₆ alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)Rₐ, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneORa, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄ alkyleneC(═O)OR^{a};
R^{a} is selected from the group consisting of hydrogen, C₁₋₆alkyl, C₃₋₈cycloalkyl, C₃₋₈ heterocycloalkyl, C₁₋₃alkyleneN(R^{c})2, aryl, arylC₁₋₃alkyl, C₁₋₃alkylenearyl, heteroaryl, heteroarylC₁₋₃alkyl, and C₁₋₃alkyleneheteroaryl;
or two R^{a} groups are taken together to form a 5- or 6-membered ring, optionally containing at least one heteroatom;
R^{b} is selected-from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

Suitable selective chemical inhibitors for use in the invention include compound having formula (II) or pharmaceutically acceptable salts and solvates thereof: 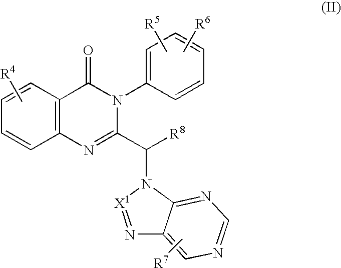
wherein R⁴, R⁵, R⁶, and R⁷, independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH2N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
R⁸ is selected from the group consisting of hydrogen, C₁₋₆alkyl, halo, CN, C(═O)R^{a}, and C(═O)OR^{a};
X¹ is selected from the group consisting of CH (i.e., a carbon atom having a hydrogen atom attached thereto) and nitrogen;
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;
or two R^{a} groups are taken together to form a 5- or 6-membered ring, optionally containing at least one heteroatom;
R^{c} is selected from the group consisting of hydrogen, C₁₋₆alkyl, C₃₋₈cycloalkyl, aryl, and heteroaryl; and,
Het is a 5- or 6-membered heterocyclic ring, saturated or partially or fully-unsaturated, containing at least one heteroatom selected from the group consisting of oxygen, nitrogen, and sulfur, and optionally substituted with C₁₋₄alkyl or C(═O)OR^{a}.

In yet another embodiment, methods of the invention include use of a PI-3-Kδ selective inhibitor compound having formula (III) or pharmaceutically acceptable salts and solvates thereof: 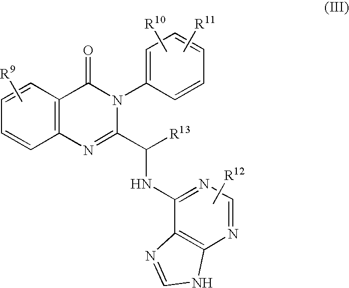
wherein R⁹, R¹⁰, R¹¹, and R¹², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃ alkyleneHet, OC₁₋₄alkyleneOC₁₋₄alkyleneC(═O)OR^{b}, C(═O)C₁₋₄alkyleneHet, and NHC(═O)haloC₁₋₆ alkyl;
R¹³ is selected from the group consisting of hydrogen, C₁₋₆alkyl, halo, CN, C(═O)R^{a}, and C(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;
or two R^{a} groups are taken together to form a 5- or 6-membered ring, optionally containing at least one heteroatom;
Rc is selected from the group consisting of hydrogen, C1_{—}6alkyl, C3_gcycloalkyl, aryl, and heteroaryl; and,
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

More specifically, methods of the invention embrace use of a PI-3-Kδ selective inhibitor selected from the group consisting of 2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2-chlorophenyl)-3H-quinazolin-4-one; 2-(6-aminopurin-o-ylmethyl)-3-(2chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7 dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9H-purin-6-yl-sulfanylmethyl)₃H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one (also known as IC87114); 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H-quinazolin-4-one; 3-cyclopropylmethyl-5-methyl-2-(9H-purin-6ylsulfanylmethyl)-3H-quinazolin-4-one; 2-(6-aminopurin-9-ylmethyl)-3cyclopropylmethyl-5-methyl-3H-quinazolin-4-one; 2-(2-amino-9H-purin-6ylsulfanylmethyl)-3-cyclopropylmethyl-5-methyl-3H-quinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclohexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6ylamino)methyl]-5-methyl-3-o-tolyl-3H-quinazolin-4-one; 2-[(2-fluoro-9H-purin-6ylamino)methyl]-5-methyl-3-o-tolyl-3H-quinazolin-4-one; (2-chlorophenyl)-dimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro-4-oxo-3,4-dihydroquinazolin-2-ylmethyl ester; N-[3-(2-chlorophenyl)-5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)-ethyl]-5-methyl-3-o-tolyl-3H-quinazolin-4-one; 5-methyl-2-[(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydropurin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5-methyl-3-o-tolyl-3H-quinazolin-4-one; 2-(4-amino-1,3,5-triazin-2-ylsulfanylmethyl)-5-methyl-3-o-tolyl-3H-quinazolin-4-one; 5-methyl-2-(7-methyl-7H-purin-6-ylsulfanylmethyl)-3-o-tolyl-3H-quinazolin 4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-Diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6ethylamino-pyrimidin-4-ylsulfanylmethyl)-5-methyl-3-o-tolyl-3H-quinazolin-4-one; 2-(3-amino-5-methylsulfanyl-1,2,4-triazol-1-ylmethyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3Hquinazolin-4-one; 3-isobutyl-5-methyl-2-(9H-purin-6-ylsulfanylmethyl)-3H-quinazolin-4-one; N-{2-[5-methyl-4-oxo-2-(9H-purin-6-ylsulfanylmethyl)-4H-quinazolin-3-yl]-phenyl}-acetamide; 5-methyl-3-(E-2-methyl-cyclohexyl)-2-(9H-purin-6-ylsulfanylmethyl)-3H-quinazolin-4-one; 2-[5-methyl-4-oxo-2-(9H-purin-6ylsulfanylmethyl)-4H-quinazolin-3-yl]-benzoic acid; 3-(2-[(2dimethylaminoethyl)methylamino]phenyl}-5-methyl-2-(9H-purin-6ylsulfanylmethyl)-3H-quinazolin-4-one; 3-(2-chlorophenyl)-5-methoxy-2-(9H-purin-6-ylsulfanylmethyl)-3H-quinazolin-4-one; -(2-chlorophenyl)-5-(2-morpholin-4-yl-ethylamino)-2-(9H-purin-6-ylsulfanylmethyl)-3H-quinazolin-4-one.

In a specific example of the methods of the invention, the PI-3-Kδ selective inhibitor 2-(6-Amino-purin-9-ylmethyl)-5-methyl-3-o-tolyl-3H-quinazolin-4-one having the chemical structure: 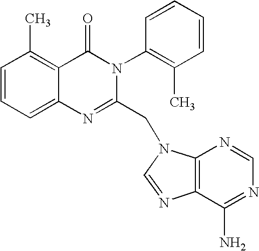
is used.

Increased understanding of these biotransformation processes permits the design of so-called “prodrugs,” which, following a biotransformation, become more physiologically active in their altered state. Prodrugs, therefore, encompass pharmacologically inactive compounds that are converted to biologically active metabolites.

To illustrate, prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound.

Compounds that compete with an inhibitor compound described herein for binding to PI-3-Kδ are also contemplated for use in the invention. Methods of identifying compounds which competitively bind with PI-3-Kδ, with respect to the compounds specifically provided herein, are well known in the art.

In view of the disclosures above, therefore, the term “inhibitor” as used herein embraces compounds disclosed, compounds that compete with disclosed compounds for PI-3-Kδ binding, and in each case, conjugates and derivatives thereof.

Inhibitors of p110δ Expression

Aspects of the invention further provides compounds that selectively negatively regulate p110δ mRNA expression more effectively than other isozymes of the PI-3-K family, and that possess acceptable pharmacological properties are contemplated for use as PI-3-Kδ selective inhibitors in the methods of the invention. Polynucleotides encoding human p110δ are disclosed, for example, in Genbank Accession Nos. AR255866, NM 005026 (see FIG. 9B), U86453, U57843 and Y10055, the disclosures of which are incorporated herein by reference in their entireties. See also, Vanhaesebroeck, et al. (1997) Proc. Natl. Acad. Sci. 94: 4330-4335, the disclosure of which is incorporated herein by reference. Representative polynucleotides encoding mouse p110δ are disclosed, for example, in Genbank Accession Nos. BC035203, AK040867, U86587, and NM_{—}008840, and a polynucleotide encoding rat p110δ is disclosed in Genback Accession No. XM_{—}345606, in each case the disclosures of which are incorporated herein by reference in their entireties.

In some aspects, the invention provides methods using antisense oligonucleotides which negatively regulate p110δ expression via hybridization to messenger RNA (mRNA) encoding p110δ. In one specific embodiment, antisense oligonucleotides at least 5 to about 50 nucleotides in length, including all lengths (measured in number of nucleotides) in between, which specifically hybridize to mRNA encoding p110δ and inhibit mRNA expression, and as a result p110δ protein expression, are contemplated by the invention. Antisense oligonucleotides include those comprising modified internucleotide linkages and/or those comprising modified nucleotides which are known in the art to improve stability of the oligonucleotide, i.e., make the oligonucleotide more resistant to nuclease degradation, particularly in vivo. It is understood in the art that, while antisense oligonucleotides that are perfectly complementary to a region in the target polynucleotide possess the highest degree of specific inhibition, antisense oligonucleotides which are not perfectly complementary, i.e., those which include a limited number of mismatches with respect to a region in the target polynucleotide, also retain high degrees of hybridization specificity and therefore inhibit expression of the target mRNA. Accordingly, the invention contemplate methods using antisense oligonucleotides that are perfectly complementary to a target region in a polynucleotide encoding p110δ, as well as methods that utilize antisense oligonucleotides that are not perfectly complementary, i.e., include mismatches, to a target region in the target polynucleotide to the extent that the mismatches do not preclude specific hybridization to the target region in the target polynucleotide. For example, preparation and use of antisense compounds are described in U.S. Pat. No. 6,277,981.

Aspects of the invention further contemplate methods utilizing ribozyme inhibitors which, as is known in the art, include a nucleotide region which specifically hybridizes to a target polynucleotide and an enzymatic moiety that digests the target polynucleotide. Specificity of ribozyme inhibition is related to the length the antisense region and the degree of complementarity of the antisense region to the target region in the target polynucleotide. These aspects of the invention therefore contemplate ribozyme inhibitors comprising antisense regions from 5 to about 50 nucleotides in length, including all nucleotide lengths in between, that are perfectly complementary, as well as antisense regions that include mismatches to the extent that the mismatches do not preclude specific hybridization to the target region in the target p110δ encoding polynucleotide. Ribozymes useful in methods of the invention include those comprising modified internucleotide linkages and/or those comprising modified nucleotides which are known in the art to improve stability of the oligonucleotide, i.e., make the oligonucleotide more resistant to nuclease degradation, particularly in vivo, to the extent that the modifications do not alter the ability of the ribozyme to specifically hybridize to the target region or diminish enzymatic activity of the molecule. Because ribozymes are enzymatic, a single molecule is able to direct digestion of multiple target molecules thereby offering the advantage of being effective at lower concentrations than non-enzymatic antisense oligonucleotides. Preparation and use of ribozyme technology are described, e.g., in U.S. Pat. Nos. 6,696,250, 6,410,224, and 5,225,347.

Aspects of the invention also contemplate use of methods in which RNAi technology is utilized for inhibiting p110δ expression. In one embodiment, the invention provides double-stranded RNA (dsRNA) wherein one strand is complementary to a target region in a target p110δ-encoding polynucleotide. In general, dsRNA molecules of this type less than 30 nucleotides in length are referred to in the art as short interfering RNA (siRNA). The invention also contemplates, however, use of dsRNA molecules longer than 30 nucleotides in length, and in certain embodiments of the invention, these longer dsRNA molecules can be about 30 nucleotides in length up to 200 nucleotides in length and longer, and including all length dsRNA molecules in between. As with other RNA inhibitors, complementarity of one strand in the dsRNA molecule can be a perfect match with the target region in the target polynucleotide, or may include mismatches to the extent that the mismatches do not preclude specific hybridization to the target region in the target p110δ-encoding polynucleotide. As with other RNA inhibition technologies, dsRNA molecules include those comprising modified internucleotide linkages and/or those comprising modified nucleotides which are known in the art to improve stability of the oligonucleotide, i.e., make the oligonucleotide more resistant to nuclease degradation, particularly in vivo. For example, preparation and use of RNAi compounds are described in U.S. Patent Application No. 20040023390.

Aspects of the invention further contemplate methods wherein inhibition of p110δ is effected using “RNA lasso” technology. Circular RNA lasso inhibitors are highly structured nucleic acid molecules that are inherently more resistant to degradation and therefore do not, in general, include or require modified internucleotide linkage or modified nucleotides. The circular lasso structure includes a region that is capable of hybridizing to a target region in a target polynucleotide, the hybridizing region in the lasso being of a length typical for other RNA inhibiting technologies. As with other RNA inhibiting technologies, the hybridizing region in the lasso may be a perfect match with the target region in the target polynucleotide, or may include mismatches to the extent that the mismatches do not preclude specific hybridization to the target region in the target p110δ-encoding polynucleotide. Because RNA lassos are circular and form tight topological linkage with the target region, inhibitors of this type are generally not displaced by helicase action unlike typical antisense oligonucleotides, and therefore can be utilized as dosages lower than typical antisense oligonucleotides. Preparation and use of RNA lassos are described, for example, in U.S. Pat. No. 6,369,038.

Pharmaceutical Formulations and Delivery

The inhibitors of the invention may be covalently or noncovalently associated with a carrier molecule, such as a linear polymer (e.g., polyethylene glycol, polylysine, dextran, etc.), a branched-chain polymer (see U.S. Pat. Nos. 4,289,872 and 5,229,490; PCT Publication WO 93121259 published 28 Oct. 1993); a lipid; a cholesterol group (such as a steroid); or a carbohydrate or oligosaccharide. Specific examples of carriers for use in the pharmaceutical compositions of the invention include carbohydrate-based polymers, such as trehalose, mannitol, xylitol, sucrose, lactose, sorbitol, dextrans, such as cyclodextran, cellulose, and cellulose derivatives. Also, the use of liposomes, microcapsules or microspheres, inclusion complexes, or other types of carriers is contemplated.

Other carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Still other useful carrier polymers known in the art include monomethoxy-polyethylene glycol, poly-(N-vinyl pyrrolidone)-polyethylene glycol, propylene glycol homopolymers, a polypropylene oxide/ethylene oxide copolymer, polyoxyethylated polyols (e.g., glycerol) and polyvinyl alcohol, as well as mixtures of these polymers.

Derivatization with bifunctional agents is useful for cross-linking a compound of the invention to a support matrix or to a carrier. One such carrier is polyethylene glycol (PEG). The PEG group may be of any convenient molecular weight and may be straight chain or branched. The average molecular weight of the PEG can range from about 2 kDa to about 100 kDa, in another aspect from about 5 kDa to about 50 kDa, and in a further aspect from about 5 kDa to about 10 kDa. The PEG groups will generally be attached to the compounds of the invention via acylation, reductive alkylation, Michael addition, thiol alkylation or other chemoselective conjugation/ligation methods through a reactive group on the PEG moiety (e.g., an aldehyde, amino, ester, thiol, haloacetyl, maleimido or hydrazino group) to a reactive group on the target inhibitor compound (e.g., an aldehyde, amino, ester, thiol, α-haloacetyl, maleimido or hydrazino group). Cross-linking agents can include, e.g., esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287, 3,691,016, 4,195,128, 4,247,642, 4,229,537, and 4,330,440 may be employed for inhibitor immobilization.

The pharmaceutical compositions of the invention may also include compounds derivatized to include one or more antibody Fc regions. Fc regions of antibodies comprise monomeric polypeptides that may be in dimeric or multimeric forms linked by disulfide bonds or by non-covalent association. The number of intermolecular disulfide bonds between monomeric subunits of Fc molecules can be from one to four depending on the class (e.g., IgG, IgA, IgE) or subclass (e.g., IgG1, IgG2, IgG3, IgA1, IgGA2) of antibody from which the Fc region is derived. The term “Fc” as used herein is generic to the monomeric, dimeric, and multimeric forms of Fc molecules, with the Fc region being a wild type structure or a derivatized structure. The pharmaceutical compositions of the invention may also include the salvage receptor binding domain of an Fc molecule as described in WO 96/32478, as well as other Fc molecules described in WO 97/34631.

Such derivatized moieties preferably improve one or more characteristics of the inhibitor compounds of the invention, including for example, biological activity, solubility, absorption, biological half life, and the like. Alternatively, derivatized moieties result in compounds that have the same, or essentially the same, characteristics and/or properties of the compound that is not derivatized. The moieties may alternatively eliminate or attenuate any undesirable side effect of the compounds and the like.

Methods include administration of an inhibitor to an individual in need, by itself, or in combination as described herein, and in each case optionally including one or more suitable diluents, fillers, salts, disintegrants, binders, lubricants, glidants, wetting agents, controlled release matrices, colorants/flavoring, carriers, excipients, buffers, stabilizers, solubilizers, other materials well known in the art and combinations thereof.

Any pharmaceutically acceptable (i.e., sterile and non-toxic) liquid, semisolid, or solid diluents known in the art that serve as pharmaceutical vehicles, excipients, or media may be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch. Some representative commercially available diluents are Fast-Flo, Emdex, STA-Rx 1500, Emcompress and Avicell. Such compositions may influence the physical state, stability, rate of in vivo release, and rate of in vivo clearance of the present inhibitor compounds. See, e.g., Remington's Pharmaceutical Sciences, 18th Ed. (1990, Mack Publishing Co., Easton, Pa. 18042) pages 1435-1712.

Pharmaceutically acceptable fillers can include, for example, lactose, microcrystalline cellulose, dicalcium phosphate, tricalcium phosphate, calcium sulfate, dextrose, mannitol, and/or sucrose.

Inorganic salts including calcium triphosphate, magnesium carbonate, and sodium chloride may also be used as fillers in the pharmaceutical compositions. Amino acids may be used, such as use in a buffer formulation of the pharmaceutical compositions.

Disintegrants may be included in solid dosage formulations of the inhibitors. Materials used as disintegrants include, but are not limited to, starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge, corn starch, potato starch, and bentonite may all be used as disintegrants in the pharmaceutical compositions. Other disintegrants include insoluble cationic exchange resins. Powdered gums such as agar, Karaya or tragacanth may be used as disintegrants and as binders. Alginic acid and its sodium salt are also useful as disintegrants.

Binders may be used to hold the therapeutic agent together to form a hard tablet and include materials from natural products such as acacia, tragacanth, starch and gelatin. Others include crystalline cellulose, cellulose derivatives such as methyl cellulose (MC), ethyl cellulose (EC) and carboxymethyl cellulose (CMC), acacia, corn starch, and/or gelatins Polyvinyl pyrrolidone (PVP) and hydroxypropylmethyl cellulose (HPMC) can both be used in alcoholic solutions to granulate the therapeutic.

An antifriction agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils, talc, and waxes. Soluble lubricants may also be used such as sodium lauryl sulfate, magnesium lauryl sulfate, polyethylene glycol of various molecular weights, Carbowax 4000 and 6000.

Glidants that improve the flow properties of the drug during formulation and to aid rearrangement during compression may also be added. Suitable glidants include, but are not limited to, starch, talc, pyrogenic silica and hydrated silicoaluminate.

To aid dissolution of the therapeutic into the aqueous environment, a surfactant might be added as a wetting agent. Natural or synthetic surfactants may be used. Surfactants may include, but are not limited to, anionic detergents such as sodium lauryl sulfate, dioctyl sodium sulfosuccinate, and dioctyl sodium sulfonate. Cationic detergents such as benzalkonium chloride and benzethonium chloride may be used. Nonionic detergents that can be used in the pharmaceutical formulations include, but are not limited to, lauromacrogol 400, polyoxyl 40 stearate, polyoxyethylene hydrogenated, castor oil 10, 50 and 60, glycerol monostearate, polysorbate 40, 60, 65 and 80, sucrose fatty acid ester, methyl cellulose and carboxymethyl cellulose. These surfactants could be present in the pharmaceutical compositions of the invention either alone or as a mixture in different ratios.

Controlled release formulation may be desirable. The inhibitors of aspects of the invention can be incorporated into an inert matrix which permits release by either diffusion or leaching mechanisms, e.g., gums. Slowly degenerating matrices may also be incorporated into the pharmaceutical formulations, e.g., alginates, polysaccharides. Another form of controlled release is a method based on the Oros therapeutic system (Alza Corp.), i.e., the drug is enclosed in a semipermeable membrane which allows water to enter and push the inhibitor compound out through a single small opening due to osmotic effects. Some enteric coatings also have a delayed release effect.

Colorants and flavoring agents may also be included in the pharmaceutical compositions. For example, the inhibitors of the invention may be formulated (such as by liposome or microsphere encapsulation) and then further contained within an edible product, such as a refrigerated beverage containing colorants and flavoring agents.

The therapeutic agent can also be administered in a film coated tablet. Nonenteric materials for use in coating the pharmaceutical compositions include, but are not limited to, methyl cellulose, ethyl cellulose, hydroxyethyl cellulose, methylhydroxy-ethyl cellulose, hydroxypropyl cellulose, hydroxypropyl-methyl cellulose, sodium carboxymethyl cellulose, povidone and polyethylene glycols. Enteric materials for use in coating the pharmaceutical compositions include, but are not limited to, esters of phthalic acid. A mix of materials may be used to provide the optimum film coating. Film coating manufacturing may be carried out in a pan coater, in a fluidized bed, or by compression coating.

Compositions can be administered in solid, semi-solid, liquid or gaseous form, or may be in dried powder, such as lyophilized form. The pharmaceutical compositions can be packaged in forms convenient for delivery, including, for example, capsules, sachets, cachets, gelatins, papers, tablets, capsules, ointments, granules, solutions, inhalants, aerosols, suppositories, pellets, pills, troches, lozenges or other forms known in the art. The type of packaging generally depends on the desired route of administration. Implantable sustained release formulations are also contemplated, as are transdermal formulations.

Methods of the invention contemplate administration of inhibitor compounds by various routes. Such pharmaceutical compositions may be for administration for injection, or for oral, nasal, transdermal or other forms of administration, including, e.g., by intravenous, intradermal, intramuscular, intramammary, intraperitoneal, intratracheal, intrathecal, intraocular, retrobulbar, intrapulmonary (e.g., aerosolized drugs) or subcutaneous injection (including depot administration for long term release e.g., embedded under the splenic capsule, brain, or in the cornea); by sublingual, anal, vaginal, placental, or by surgical implantation, e.g., embedded under the splenic capsule, brain, or in the cornea. The treatment may consist of a single dose or a plurality of doses over a period of time. In general, the methods of the invention involve administering effective amounts of an inhibitor of the invention together with pharmaceutically acceptable diluents, preservatives, solubilizers, emulsifiers, adjuvants and/or carriers, as described above. As is understood in the art, a chosen route of administration may dictate the physical form of the compound being delivered.

In one aspect, the invention provides methods for oral administration of a pharmaceutical composition of the invention. Oral solid sage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, and cachets or pellets. Also, liposomal or proteinoid encapsulation maybe used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation includes a compound of the invention and inert ingredients which protect against degradation in the stomach and which permit release of the biologically active material in the intestine.

The inhibitors can be included in the formulation as fine multiparticulates in the form of granules or pellets of particle size about 1 mm. The formulation of the material for capsule administration could also be as a powder, lightly compressed plugs or even as tablets. The capsules could be prepared by compression.

Also contemplated herein is pulmonary delivery of the present inhibitors in accordance with the invention. According to this aspect of the invention, the inhibitor is delivered to the lungs of a mammal while inhaling and traverses across the lung epithelial lining to the blood stream.

Contemplated for use in the practice of aspects of this invention are a wide range of mechanical devices designed for pulmonary delivery of therapeutic products, including, but not limited to, nebulizers, metered dose inhalers, and powder inhalers, all of which are familiar to those skilled in the art. Some non-limited examples of commercially available devices suitable for the practice of this invention are the Ultravent nebulizer, manufactured by Mallinckrodt, Inc., St. Louis, Mo.; the Acorn H nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

When used in pulmonary administration methods, the inventive inhibitors are most advantageously prepared in particulate form with an average particle size of less than 10 μm (or microns), for example, 0.5 μm to 5 μm, for most effective delivery to the distal lung.

Formulations suitable for use with a nebulizer, either jet or ultrasonic, will typically comprise the inventive compound dissolved in water at a concentration range of about 0.1 mg to 100 mg of inhibitor per mL of solution, 1 mg to 50 mg of inhibitor per mL of solution, or 5 mg to 25 mg of inhibitor per mL of solution. The formulation may also include a buffer. The nebulizer formulation may also contain a surfactant, to reduce or prevent surface induced aggregation of the inhibitor caused by atomization of the solution in forming the aerosol.

Formulations for use with a metered-dose inhaler device generally comprise a finely divided powder containing the inventive inhibitors suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device generally comprise a finely divided dry powder containing the inventive compound and may also include a bulking agent or diluent, such as lactose, sorbitol, sucrose, mannitol, trehalose, or xylitol in amounts which facilitate dispersal of the powder from the device, e.g., 50 to 90% by weight of the formulation.

Nasal delivery of the inventive compound is also contemplated. Nasal delivery allows the passage of the inhibitor to the blood stream directly after administering the therapeutic product to the nose, without the necessity for deposition of the product in the lung. Formulations for nasal delivery may include dextran or cyclodextran. Delivery via transport across other mucous membranes is also contemplated.

In practice of the methods of the inventions, the pharmaceutical compositions are generally provided in doses ranging from 1 pg compound/kg body weight to 1000 mg/kg, 0.1 mg/kg to 100 mg/kg to 50 mg/kg, and 1 to 20 mg/kg, given in daily doses or in equivalent doses at longer or shorter intervals, e.g., every other day, twice weekly, weekly, or twice or three times daily. The inhibitor compositions may be administered by an initial bolus followed by a continuous infusion to maintain therapeutic circulating levels of drug product. Those of ordinary skill in the art will readily optimize effective dosages and administration regimens as determined by good medical practice and the clinical condition of the individual patient. The frequency of dosing will depend on the pharmacokinetic parameters of the agents and the route of administration. The optimal pharmaceutical formulation will be determined by one skilled in the art depending upon the route of administration and desired dosage. See for example, Remington's Pharmaceutical Sciences, 18th Ed. (1990, Mack Publishing Co., Easton, Pa. 18042) pages 1435-1712, the disclosure of which is hereby incorporated by reference. Such formulations may influence the physical state, stability, rate of in vivo release, and rate of in vivo clearance of the administered agents. Depending on the route of administration, a suitable dose may be calculated according to body weight, body surface area or organ size. Further refinement of the calculations necessary to determine the appropriate dosage for treatment involving each of the above mentioned formulations is routinely made by those of ordinary skill in the art without undue experimentation, especially in light of the dosage information and assays disclosed herein, as well as the pharmacokinetic data observed in the human clinical trials discussed above. Appropriate dosages may be ascertained through use of established assays for determining blood levels dosages in conjunction with appropriate physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. As studies are conducted, further information will emerge regarding the appropriate dosage levels and duration of treatment for various diseases and conditions.

### EXAMPLES

The following examples are provided to illustrate the invention, but are not intended to limit the scope thereof.

### Example 1

### Preparation of a Hypertensive Animal Model and Evidence that PI-3-K Plays a Role in Arterial Spontaneous Tone

Previous studies examining alterations in PI-3-kinase-mediated spontaneous tone used the aorta as the vessel of choice (Northcott, et al., (2002) Circ Res. 91: 360-369). The aorta is a conduit artery and has been found to play at least a small role in the maintenance of blood pressure, due to changes in compliance in the aorta during the condition of hypertension (Safar, et al. (1998) Hypertension 32: 156-161; Salaymeh and Banerjee (2001) Am. Heart J., 142: 549-555). The function of resistance arteries, however, is more immediately relevant to control of TPR, because small changes in the diameter of resistance arteries can lead to large changes of TPR due to their relationship (resistance, R, is proportional to 1/r⁴). A series of experiments were therefore designed to determine if PI-3-kinase participates in the resistance artery control.

Male Sprague Dawley rats (250-300 g; Charles River Laboratories, Inc., Portage, Mich.) were made hypertensive as follows. In brief, individual rats underwent uninephrectomy and implantation of deoxycorticosterone acetate (DOCA; 200 mg/kg) under isoflurane anesthesia as described previously (Florian et al. (1999) Am. J. Physiol. 276: H976-H983). Animals remained on the regimen for four weeks, after which time systolic blood pressures were measured using standard tail cuff methods. Results indicated that the systolic blood pressure of the DOCA-salt and sham rats were 190±3 mm Hg and 121±2 mm Hg, respectively.

Resistance arteries, approximately 240 microns in diameter, were placed in a myograph for measurements of isometric force. In brief, small mesenteric resistance arteries (2-3 mm long, 200-300 μdiameter) were dissected away from mesenteric veins under a light microscope and mounted between two tungsten wires in a dual chamber wire myograph (University of Vermont Instrumentation Shop) for measurement of isometric force. Arteries were bathed in aerated (95% O₂/5% CO₂) physiological salt solution (PSS) (37° C.) and equilibrated for 30 minutes with frequent changes of buffer prior to applying optimal tension. Optimal tension (400 mgs) was applied by means of a micrometer and the tissues were equilibrated for 60 min before exposure to a maximal concentration of phenylephrine (PE, Sigma Chemical Co, St. Louis, Mo.) (10⁻⁵ mol/L). Spontaneous tone was monitored, LY294002 (Biomol, Plymouth Meeting, Pa.) (20 μmol/L) or vehicle (0.1% DMSO) was added for 30 minutes, and the change in tone was recorded.

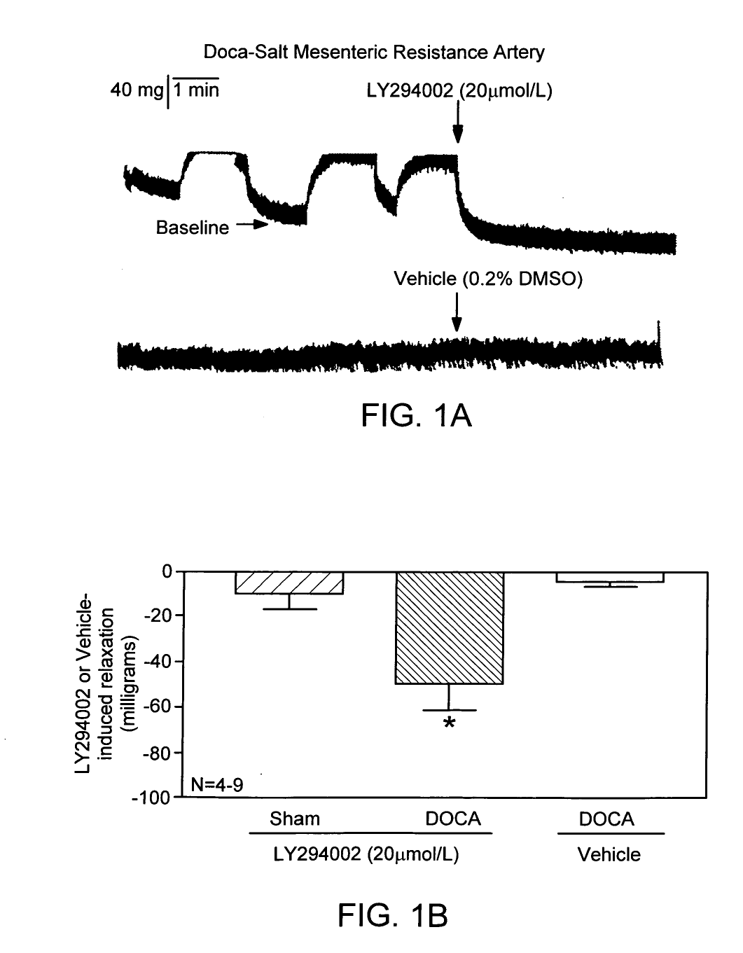

Results showed that elevated tone developed in several of the resistance arteries removed from the DOCA-salt rats. Spontaneous tone did not develop in resistance arteries removed from sham rats. LY294002 (20 μmol/L) significantly inhibited tone in the resistance arteries from DOCA-salt rats as compared to sham or vehicle-incubated arteries from DOCA-salt rats (see FIGS. 1A and B). FIG. 1A shows a representative tracing of spontaneous arterial tone in endothelium-denuded mesenteric resistance arteries from DOCA-salt treated rat (200 to 300 μm in diameter). Tissues were under passive tension for optimal force production; vehicle (0.1% DMSO) or LY294002 (20 μmol/L) was added and allowed to equilibrate for 1 hour. The arrow represents the baseline at which quantification of the LY294002-induced relaxation was compared. FIG. 1B shows the effect of PI-3-kinase inhibitor LY294002 or vehicle on spontaneous tone in endothelium-denuded rat aorta from DOCA-salt and sham rats. Bars represent the LY294002 or vehicle-induced relaxation (milligrams) in the mesenteric resistance arteries±SEM (* denotes a statistically significant difference (P<0.05) between DOCA-salt vehicle and LY294002 treatment groups. Because LY294002 had no effect on nor did spontaneous tone develop in resistance arteries and aorta from sham rats, changes in PI-3-kinase activity were specific to the arteries from hypertensive animals.

### Example 2

### Biochemical Analysis of Arterial Proteins in Hypertensive Animals

In view of the results obtained in Example 1 showing inhibition of PI-3-kinase inhibited tone development in hypertensive animals, biochemical analyses were carried out to specifically characterize the PI-3-kinase activity.

Mesenteric resistance arteries were cleaned, pooled, quick-frozen, pulverized in liquid nitrogen-cooled mortar and solubilized in lysis buffer [0.5 mol/L Tris HCl (pH 6.8), 10% SDS, 10% glycerol] with protease inhibitors (0.5 mmol/L PMSF, 10 μg/ml aprotinin and 10 pg/ml leupeptin). Homogenates were centrifuged (11,000 g for 15 min, 4° C.) and supernatant total protein measured. Equivalent amounts of mesenteric resistance arterial protein from sham and DOCA-salt rats were separated on 7% SDS-polyacrylamide gels and transferred to Immobilon-P membrane for standard western analyses using anti-p85α (1:100; Upstate Biotechnology, Lake Placid, N.Y.), anti-p110δ (1:1000; Santa Cruz Biotechnology, Inc., Santa Cruz, Calif.), anti-Akt and anti-pAkt (1:1000; Cell Signaling, Beverly, Mass.) antibodies. Anti-smooth muscle O-actin (1:400; Oncogene, Cambridge, Mass.) was used to normalize protein to smooth muscle content.

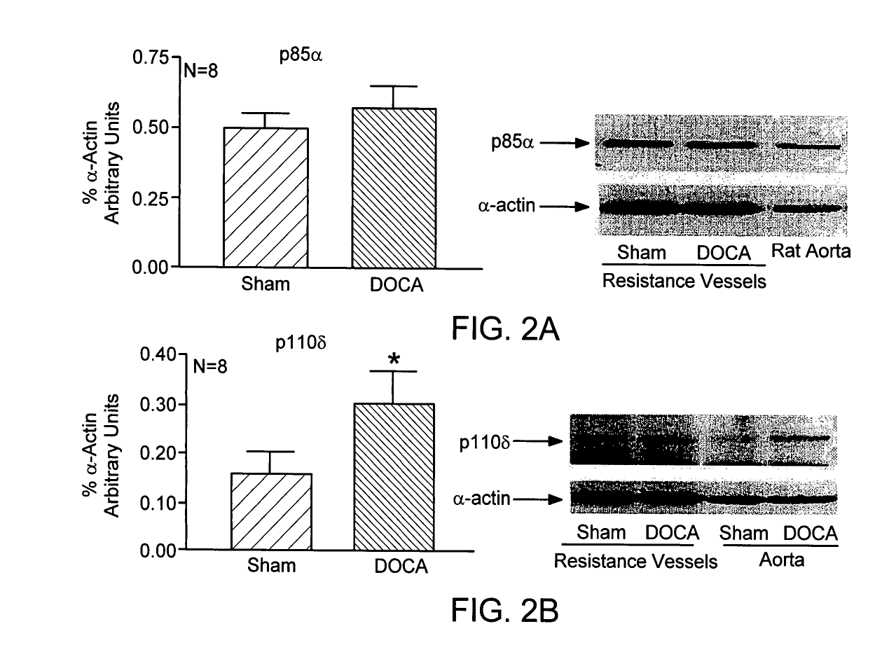

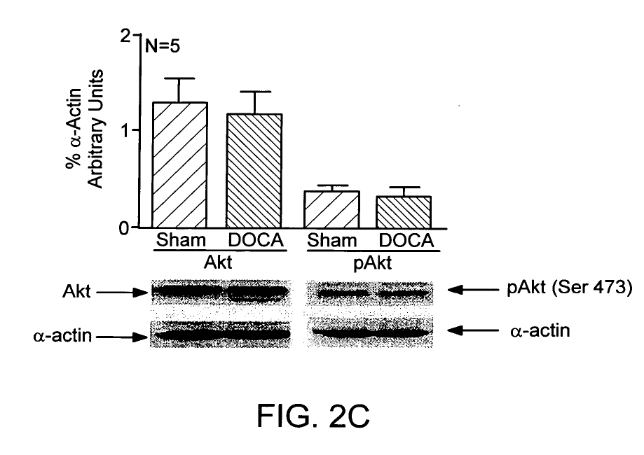

Western analyses revealed the presence of p85α, p110δ, Akt and pAkt protein in resistance arteries from both sham and DOCA-salt rats (see FIGS. 2A-2C). FIG. 2 shows Western blot analyses of protein isolated from mesenteric resistance arteries from sham and DOCA-salt-treated rats using antibodies specific for p85α (FIG. 2A), p110δ (FIG. 2B), and Akt/pAkt (FIG. 2C) (Bars represent mean arbitrary densitometry units ±SEM; and * indicates a statistically difference (P<0.05) between sham and DOCA-salt treatment groups). Rat aortic controls were run as positive controls for the respective antibodies. Akt is a signaling enzyme phosphorylated by PI-3-kinase and is commonly used to examine PI-3-kinase activity in cells. There was significantly greater Class IA catalytic PI-3-kinase subunit p 1106 protein in resistance arteries from DOCA-salt rats compared to sham, however no differences were found between vessels from sham and DOCA-salt rats with respect to the p85α, Akt and pAkt protein. Results showed that, similar to the aorta, a significant increase in the p110δ subunit was observed in resistance arteries from DOCA-salt hypertensive rats (FIG. 2B). Moreover, there was no increase in p85α, Akt and pAkt in mesenteric arteries from DOCA-salt rats compared to sham (FIGS. 2A and 2C); this observation was also made in aorta (Northcott, et al., (2002) Circ Res. 91: 360-369). These studies further suggest that phosphorylation of Akt may not be an absolute measure of changes in PI-3-kinase activity, as PI-3-kinase may have targets independent of Akt. Collectively, these results further demonstrate that PI-3-kinase is a key component in spontaneous tone development in small as well as large arteries from DOCA-salt rats, suggesting PI-3-kinase plays a crucial role in hypertension-related elevated tone.

### Example 3

### Immunohistochemical Analysis of Hypertensive Arteries

To further characterize the unexpected expression of p110δ protein in vascular tissue, immunohistochemical studies were carried out to determine if p110δ expression occurred specifically in aortic vascular smooth muscle cells (VSMCs).

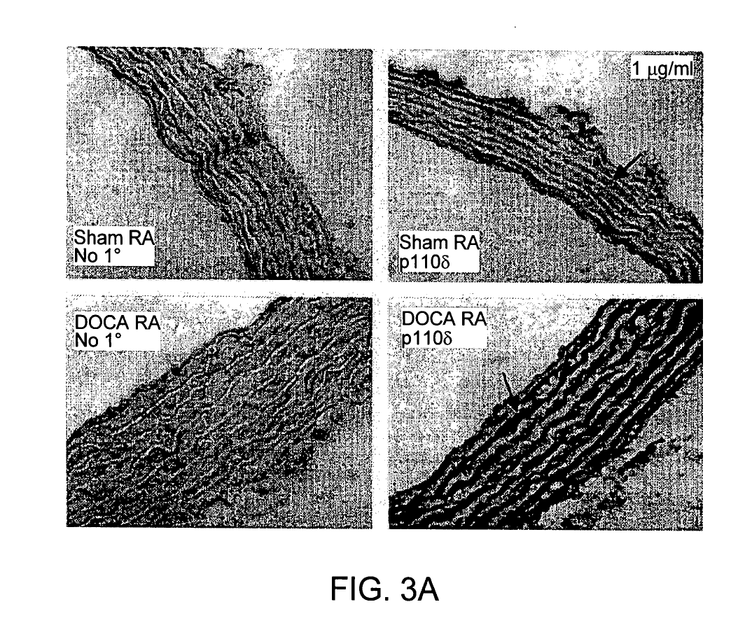

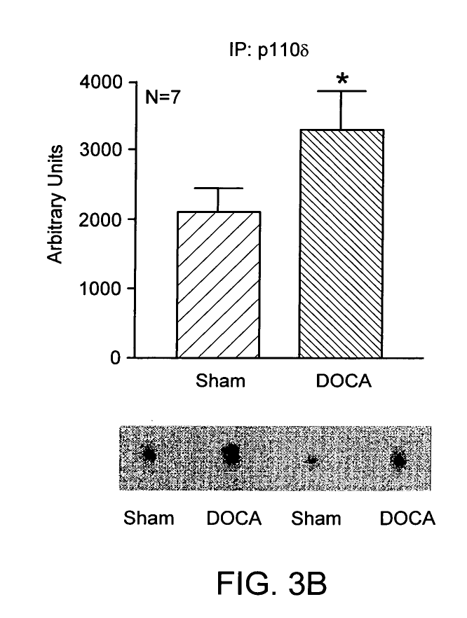

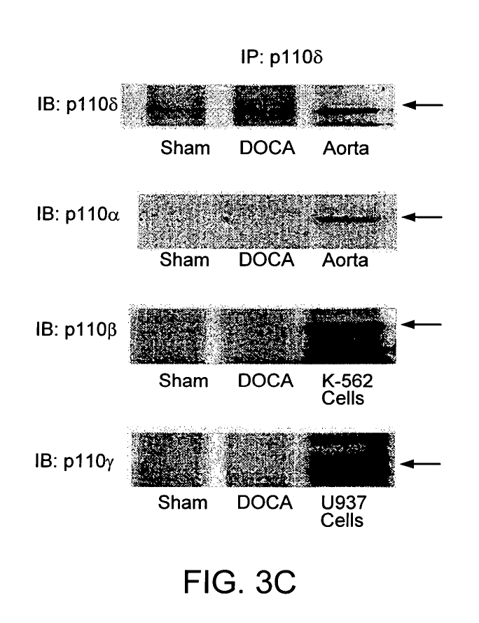

Immunohistochemistry revealed p110δ specific staining in the smooth muscle cell region in the aortae of both the sham and DOCA-salt rats (n=4) (see arrows in FIG. 3A). FIG. 3A shows representative images from immunohistochemical studies of thoracic aortae (RA) from hypertensive DOCA-salt and normotensive sham rats 8 μm sections of aorta were probed with no primary antibody (top left and bottom left) or 1 μg/ml of p110δ antibody (top right and bottom right). The arrows indicate the staining in the smooth muscle cell region of the section of those with primary antibody (note those with no primary antibody have little or no staining). The aorta from the DOCA-salt rat had more intense staining than that of the sham, supporting the increase in p110δ protein observed in aorta from DOCA-salt rat.

To further investigate the involvement of PI-3-kinase p110δ subunits in enhanced aortic PI-3-kinase activity, p110δ-specific PI-3-kinase activity assays were performed as follows. Briefly, rat thoracic aorta were cleaned as stated above, pulverized in liquid nitrogen cooled mortar and solubilized in PI-3-kinase lysis buffer. The p110δ antibody (5 μl) and protein A agarose beads (70 μl) were added to equal amounts of total protein and the samples rocked (4° C.) for 2 hours.

The PI-3-kinase assay was performed as previously described (Florian and Watts (1999) Am. J. Physiol., 276: H976-H983; Kido, et al. (2000) J. Clin. Invest., 105: 199205; Poy, et al. (2002), J. Biol. Chem., 277: 1076-1084) Briefly, the immunoprecipitated p 1103 from aortic homogenates from DOCA-salt and sham rats were incubated with phosphatidylinositol (PI) in the presence of [³²P] adenosine triphosphate (ATP). Reactions were terminated with 15 μl 4 N HCL and phospholipids extracted with 130 μl CHCl₃/methanol (1:1). The radioactive product of the reaction (PI-3-monophosphate) was detected using thin layer chromatography (TLC) and quantified with Biorad® and NIH image (v.1.61) software.

Results showed a significant increase in p110δ-associated PI-3-kinase activity in the aorta from the DOCA-salt rat compared to the sham (158% of sham) (FIG. 3B). FIG. 3B shows the presence of p110δ-associated PI-3-kinase activity in aorta from hypertensive DOCA-salt and normotensive sham rats. PI(3)P was detected using thin-layer chromatography and quantified with NIH imaging software (bars represent mean arbitrary units±SEM, and * indicates a statistically significant difference (P<0.05) between sham and DOCA-salt treatment groups). Immunoprecipitation with the p110δ antibody confirmed that the antibody reacted only to the p110δ subunit and no other p110 subunits (see FIG. 3C). FIG. 3C shows the results of immunoprecipitation (IP) with p110δ, antibody of aortic lysates from hypertensive DOCA-salt and normotensive sham rats to examine if any of the other p110 subunits could react to the p110δ antibody. Bots were immunoblotted (IB) with antibodies against p110α, p110α, p110β, and p110γ. Only aortic samples immoblotted for p110δ showed positive staining for the antibody, suggesting specificity for the p110δ antibody in immunoprecipitation (rat aortic lysate, K-562, or U937 cellular lysates were ran as positive controls for the antibodies used). This observation provided support to the hypothesis that increased p110δ PI-3-kinase activity mediates enhanced p110δ-mediated tone in aorta from DOCA-salt rats.

### Example 4

### Evidence for Role of p110δ in Spontaneous Tone Development

In order to determine if the PI-3-kinase role in tone development could be ascribed to a specific subunit(s), myography was carried out using a p110 subunit specific inhibitor (IC87114).

Endothelial cell-denuded thoracic aorta, removed from pentobarbital (60 mg kg⁻¹, i.p.) anesthetized rats, were pair-mounted (Sham/DOCA) in isolated tissue baths for measurement of isometric force. (Florian and Watts (1999) Am. J. Physiol., 276: H976-H983) Tissues were challenged with a maximal concentration of a adrenergic agonist, phenylephrine (PE) (10⁻⁵ mol/L). IC87114 (ICOS Corporation, Bothell, Wash.) concentration response curves were generated by adding increasing concentrations of IC87114 (1×10⁻⁹-3×10⁻⁴ mol/L) with measurements of spontaneous tone taken every 30 minutes. Aortic strips from DOCA-salt rats were also exposed to 20 μmol/L IC87114 or vehicle for 1 hour and measurements of spontaneous tone were recorded.

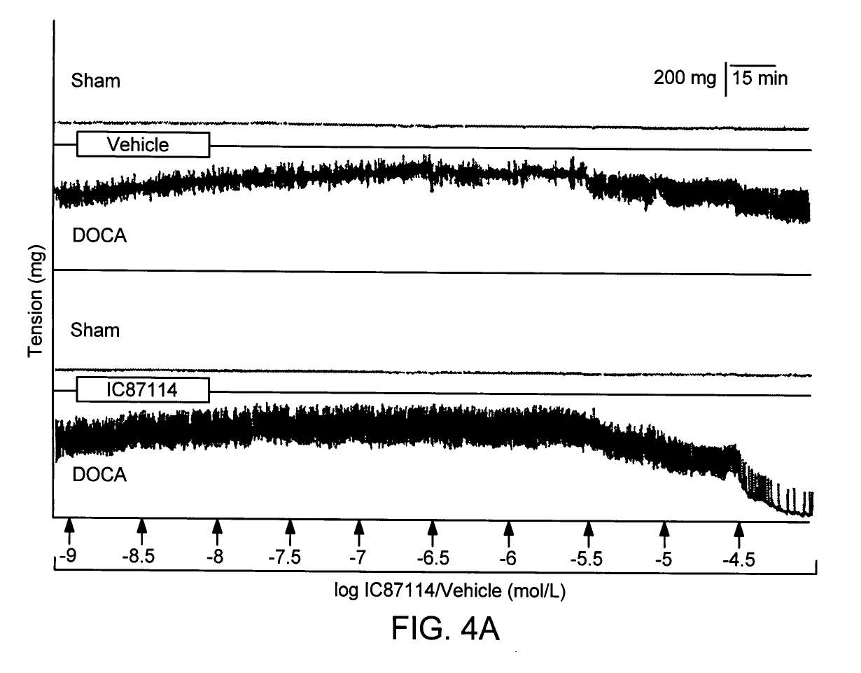

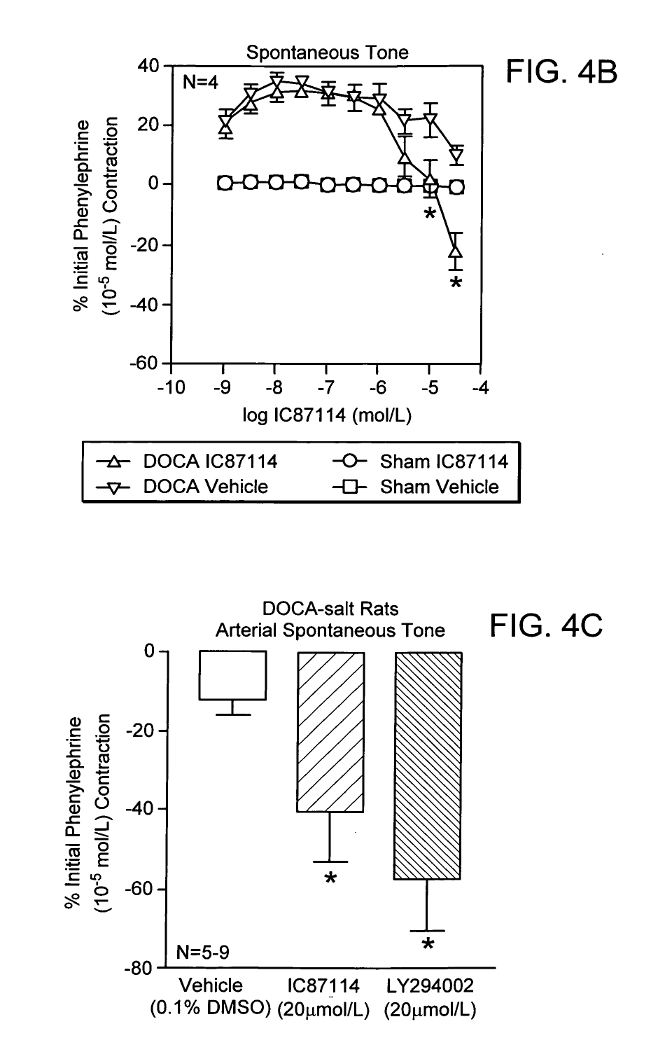

Results showed that spontaneous tone developed in aorta from DOCA-salt but not sham rats (see FIGS. 4A and 4B). FIG. 4A shows reprentative tracings of vehicle and IC87114 (1×10⁻⁹ to 3×10⁻⁵ mol/L) concentration response curves to endothelium-denuded aorta from DOCA-salt and sham rats. Tissues were under passive tension for optimal force production. FIG. 4B shows the effect of increasing concentrations of IC87114 or vehicle on spontaneous tone in aorta from DOCA-salt and control rats (points represent ±SEM). When increasing concentrations of IC87114 (10⁻⁹ to 3×10⁻⁴ mol/L) or vehicle (DMSO) was added to endothelium-denuded aortic strips from DOCA-salt rats in the absence of agonist, IC87114 reduced spontaneous tone in a concentration-dependent manner and at concentrations that do not significantly affect the other p110 subunits present in the aorta. The effect of IC87114 was reversible in all experiments, as spontaneous tone was restored upon washing out of IC87114.

In further experiments using an IC87114 concentration equivalent to that used in previous experiments with LY294002 (20 μmol/L) (Example 1), IC87114 (20 μmol/L) or vehicle (0.1% DMSO) was incubated with aortic strips from DOCA-salt rats for 1 hour in isolated tissue baths. Results further demonstrated that IC87114 significantly inhibits spontaneous tone development in DOCA-salt rats compared to vehicle (FIG. 4C). FIG. 4C shows the effect of IC81174 (20 mmol/L), LY294002 (20 mmol/L), or vehicle (0.1% DMSO), incubated for one hour, on spontaneous tone in aorta from DOCA-salt treated and control rats (data are presented as a percentage of the initial phenylephrine (PE) (10⁻⁵ mol/L) contraction; bars represent means±SEM, and * indicates a statistically significant difference (P<0.05) between DOCA-salt vehicle and treatment groups).

These data support an increase in PI-3-kinase-mediated spontaneous tone and an increase in PI-3-kinase protein, specifically the p110δ subunit in the mesenteric resistance arteries. These data therefore emphasize the critical importance of the p110δ PI-3-kinase subunit to the development of hypertension and hypertension-related conditions by showing that it is localized to VSMC, upregulated in both activity and expression, and pharmacologically-responsive to specific inhibitors as evidenced by changes in spontaneous tone.

### Example 5

### Animal Model for Genetically-Based Hypertension and Evidence for Involvement of PI-3-K

Genetically-based hypertension, as exemplified in the spontaneously hypertensive rat (SHR), is more common than a mineralocorticoid-based form of hypertension. Thus it is important to further demonstrate test that PI-3-K is a key mediator of spontaneous tone and hypercontractility in genetically-based. Arterial hypercontractility is a hallmark of hypertension that is observed in both experimental and genetically-based forms of hypertension. The following experiments demonstrate that two particular forms of hypercontractility, i.e., spontaneous tone and supersensitivity to contractile agonists, depend upon the enzyme PI-3-K. In particular, the results described show that arteries from genetically hypertensive (SHR) rats display both forms of hypercontractility and that PI-3-K function is important to each.

In order to demonstrate that PI-3-K activity is involved in the etiology of genetically-based hypertension, the systolic blood pressures of normal WKY rats (11-14 weeks old) and hypertensive SHR rats (12 weeks old) were first compared. Briefly, both WKY and SHR rats were obtained from Taconic Farmers, Inc. (Germantown, N.Y.). Systolic blood pressures of conscious rats were determined by the tail cuff method using a pneumatic transducer. Three blood pressure measurements were taken to obtain an average measurement. The results showed that the blood pressure of the genetically hypertensive SHR was significantly higher (175±9 mm Hg; N=6) than that of the normotensive WKY rat controls (114±3 mm Hg; N=6).

To further demonstrate that this difference in blood pressure measurement was associated with a PI-3-K mediated difference in aortic spontaneous tone, the spontaneous tone of aortas from normal and hypertensive rats in the presence and absence of PI-3-K inhibitor was examined. Briefly, Rats were euthanized using 60 mg kg-1 pentobarbital (ip). Aortae were removed, placed in physiological salt solution (PSS, mM) (103 NaCl; 4.7 KCl; 1.18 KH₂PO₄; 1.17 MgSO₄.7H₂O; 1.6 CaCl₂-2H₂O; 14.9 NaHCO₃; 5.5 dextrose, and 0.03 CaNa₂ EDTA), cleaned of fat and connective tissue and cut into helical strips. The endothelium was removed by gently rubbing the luminal face with a moistened cotton swab. Two paired strips (one WKY, one SHR) were mounted in 10 ml tissue baths for isometric tension recordings using Grass® force-displacement transducer FT03C (Grass Instruments, Quincy, Mass.) connected to a PowerLab/s v.3.6 and Chart v.3.6.3/s software (Mountain View, Calif.). Tissue baths contained warmed (37° C.), aerated (95% O₂/CO₂)PSS. Strips were placed under optimum resting tension (1,500 mg for aorta, determined previously), equilibrated for one hour and challenged initially with a maximal concentration of the a1-adrenergic agonist, phenylephrine (PE; 10 mM). Tissues were washed and tested for the removal of the endothelial cells by examining endothelium-dependent relaxation to acetylcholine (ACh) (1 mM) in strips contracted to a half-maximal concentration of PE. Strips relaxed <5% to ACh and were considered denuded of functional endothelial cells. Cumulative concentration curves were performed to NE (10⁻⁹-3×10⁻⁵ M). LY294002 (20 μM) or vehicle (0.02% DMSO) were incubated with the vessels for 30 minutes prior to experimentation. Spontaneous tone was defined as a change in arterial tone independent of exogenous stimulus that was a steady increase in arterial tone, not phasic or oscillatory changes. After the endothelial cell integrity test, tissues rested for one hour with washes every 10 minutes. During this time, spontaneous tone was measured. At this point, vehicle (DMSO) or LY294002 (20 μM) was added for 30 minutes and alterations in tone recorded.

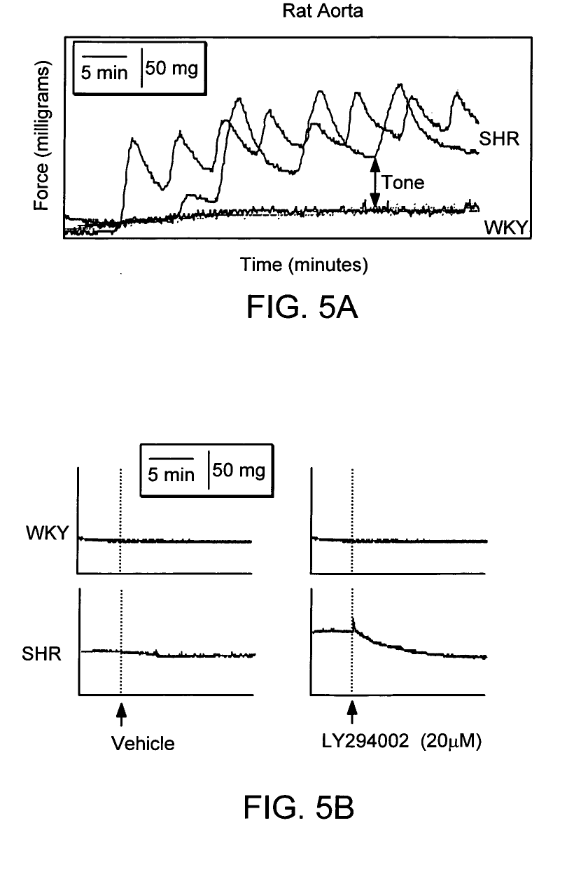

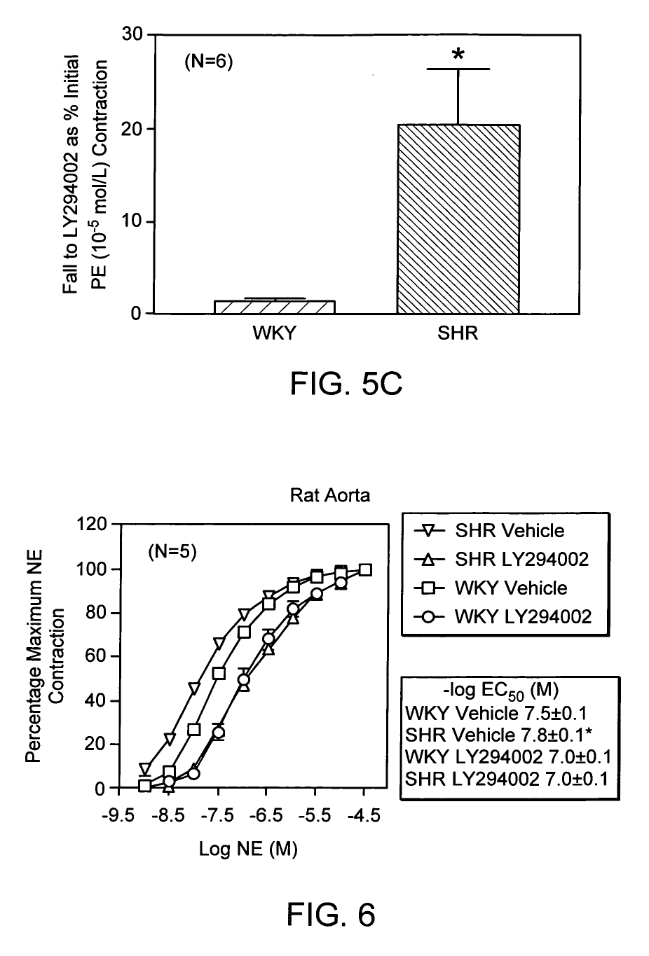

The results show that spontaneous tone occurred in the endothelium-denuded aorta isolated from the hypertensive SHR, while tone was not observed in aorta from normotensive WKY rats (FIG. 5A, marked tone). FIG. 5A shows an example of spontaneous tone in strips from two different SHR rats compared to WKY. Spontaneous tone is the stable, tonic contraction that underlies the phasic oscillatory contractions that are present. The non-selective PI-3-K inhibitor LY294002 (20 μM) caused a significant decrease in basal tone of the aorta from the SHR as compared to WKY (FIG. 5B) while vehicle had minimal effect in either group. FIG. 5B shows the effect of vehicle (left) and LY294002 (right; 20 mM) on basal tone in WKY (top) and SHR (bottom) aortic strips. The fall in basal tone to LY294002 was quantified as a percentage of the initial response to PE in FIG. 5C. LY294002 caused a significantly greater magnitude decrease in basal tone compared to WKY. FIG. 5C shows a quantification of the magnitude of reduction in basal tone caused by LY294002 (20 mM) in aortic strips from WKY and SHR animals (bars represent means±SEM for the number of animals indicated by N, and the * indicate statistically significant differences (P<0.05) between WKY and SHR values. These results support the involvement of PI-3-K in the etiology of genetically based hypertension.

### Example 6

### Evidence for Involvement of PI-3-K in NE-Induced Contraction

The effect of LY294002 on NE-induced contraction was next examined. The concentration response curve to NE in aorta from SHR was significantly leftward shifted as compared to its normotensive WKY control, and the threshold concentration of NE to cause contraction was significantly lower in SHR compared to WKY (FIG. 6). FIG. 6 shows the effect of vehicle or LY294002 (20 mM) on NE-induced contraction in aortic strips from WKY and SHR animals (the * indicate statistically significant differences from WKY vehicle). Potency values of NE (−log EC₅₀) were calculated using an algorithm in GraphPad Prism®. Points represent means±SEM for number of animals indicated by N. The results show that, in the presence of LY294002, NE-induced contraction was rightward shifted in the WKY and SHR compared to vehicle treated control tissues. The EC₅₀ values of the LY294002-incubated tissues were not significantly different, evidence that LY294002 normalized the hyperresponsiveness to NE in the aorta from SHR.

### Example 7

### Quantitative Biochemical Analysis of PI-3-K Signaling Pathway

One potential reason for an increase in apparent function of PI-3-K is increased expression of the enzyme. In order to examine this possibility, aorta from WKY and SHR were processed for Western detection of expression of proteins relevant to the PI-3-K signaling pathway and, where, possible, a measure of their activity. These proteins include the regulatory subunit p85α, the catalytic subunits p110α, p110β, p110γ, p110δ, downstream Akt and a PI-3-K specific phosphatase and tensin homolog (PTEN).

Briefly, in order to perform Western Analysis on these proteins, rat thoracic aortas were removed, placed in PSS and cleaned as described above. Tissues were quick frozen and pulverized in a liquid nitrogen-cooled mortar and pestle and solubilized in lysis buffer (0.5 M Tris HCl (pH 6.8), 10% SDS, 10% glycerol) with protease inhibitors (0.5 mM Phenylmethylsulfonyl fluoride (PMSF), 10 μg/μl aprotinin and 10 μg/ml leupeptin). Homogenates were centrifuged (11,000 g for 10 minutes, 4° C.) and supernatant total protein was measured using the Bicinchoninic Acid method (BCA, Sigma Chemical Co., St. Louis, Mo.). Equivalent amounts of total protein lysate containing 4:1 denaturing sample buffer was boiled for 5 minutes and separated on 10% SDS-polyacrylamide gels. Samples were electrically transferred to Immobilon PVDF membrane, blots blocked for 3 hours (4% chick egg ovalbumin, 2.5% sodium azide), and probed overnight with primary antibodies p85α (1:100, Upstate Biotechnology, Lake Placid, N.Y.), p110a (1:250; BD Transduction Laboratories, Palo Alto, Calif.), p110b, p110g, p110d (1:1000; Santa Cruz Biotechnologies, Inc.), PTEN, pPTEN, Akt, pAkt, (1:1000; Cell Signaling, Beverly, Mass.) and smooth muscle α-actin (1:400; Oncogene, San Diego, Calif.) at 4° C. Smooth muscle α-actin was used as a comparative smooth muscle cell measure, and these antibodies have been tested previously with the appropriate positive controls (Northcott et al. (2002) Circ. Res. 91: 360-69). Blots were washed and incubated with the appropriate species-specific secondary antibodies for 1 hour at 4° C. Blots were washed again and enhanced chemiluminescence was performed with ECL® reagents (Amersham Biosciences, Piscataway, N.J.) to visualize the bands.

Statistical analysis of the Western blot data are presented as means±standard error of the mean for the number of animals (N) stated. Contraction is reported as force (milligrams), as a percentage of response to maximum contraction to PE, or as a percentage of maximum contraction. EC₅₀ values (agonist concentration necessary to produce a half-maximal response) were determined using non-linear regression analysis in Prism® and reported as the mean of the negative logarithm (−log) of the EC50 value. Band density from Western analysis was quantified using the NIH imaging Version 1.61 software. When comparing two groups, the appropriate Student's t-test was used. For multiple comparisons, an ANOVA followed by Least Significant Difference analysis (LSD) and Student-Newman-Keul's (SNK) post hoc tests were performed using SAS version 8.2 statistical software. In all cases, a P value less than or equal to 0.05 was considered statistically significant.

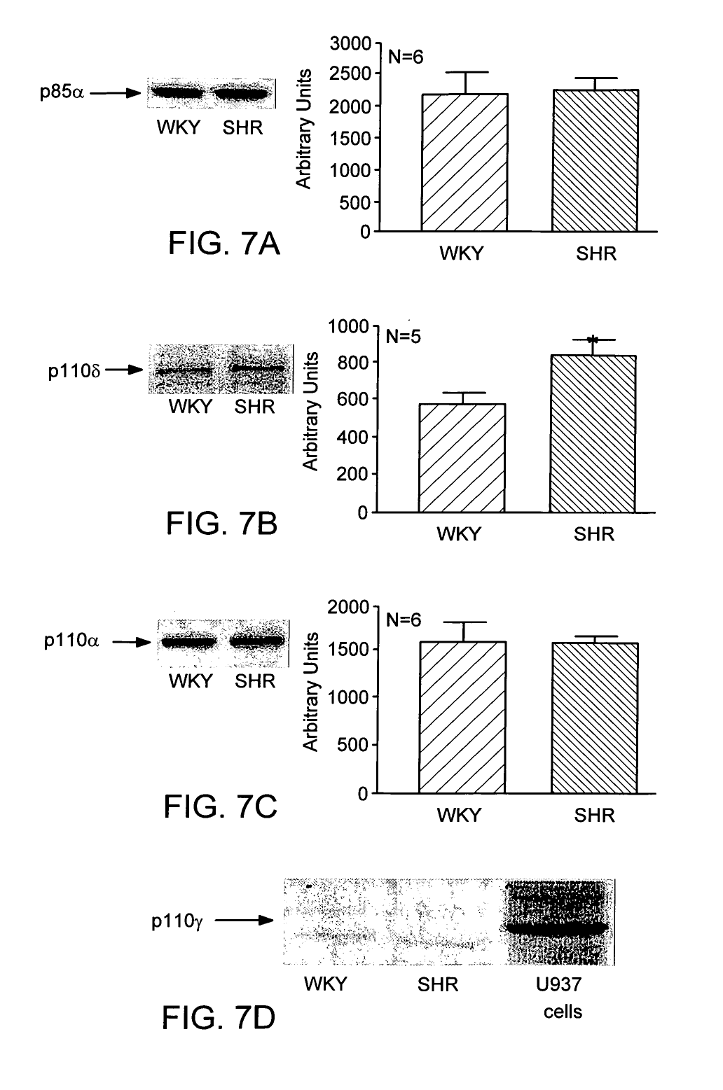

The results of the Western blot quantitative analysis for the regulatory and catalytic PI-3-K subunits are shown in FIG. 7. FIG. 7 shows sample blots and densitometry results from Western analyses probing for aortic expression of the regulatory subunit p85α (FIG. 7A), p110δ (FIG. 7B), p110α (FIG. 7C) and p110γ (FIG. 7D; U937 positive control) (bars indicated means±SEM for number of animals in parentheses, and the * indicates statistically significant differences from WKY values). The regulatory subunit p85a and catalytic p110δ and p110α PI-3-K subunits were detected. The p110γ subunit was not detected (FIG. 7D) and the p110β subunit was difficult to detect (results not shown). Importantly, there was a significantly higher p110δ protein expression in the aorta from the SHR as compared the WKY (FIG. 7B). Therefore, as with DOCA-salt induced hypertension, genetically-based hypertension in SHR rats is associated with a specific increase in the p110δ, but not other forms of p110δ from PI-3-K.

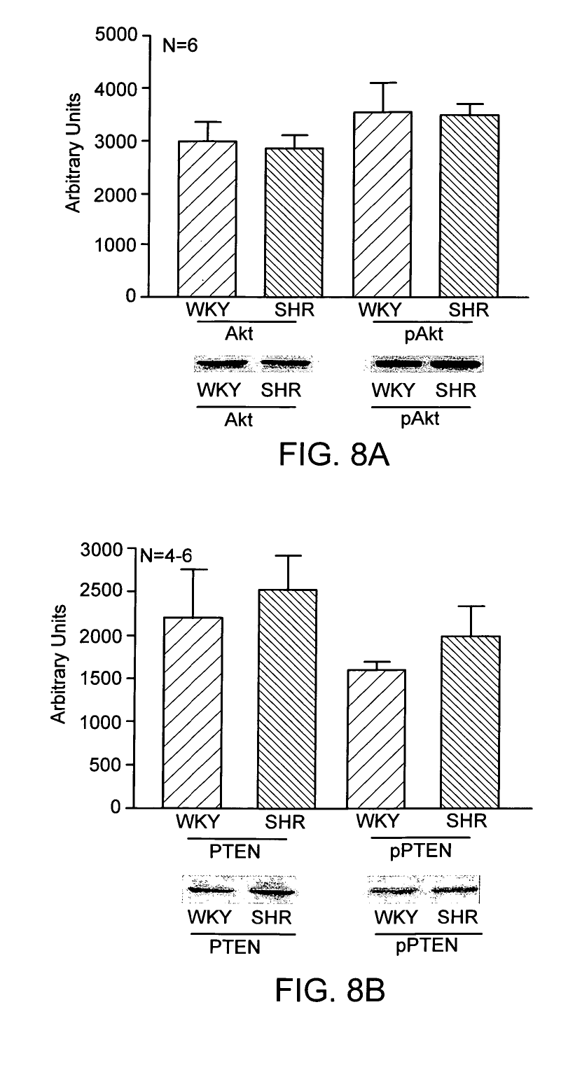

The effect of genetically based hypertension on the levels of signaling factors downstream of the PI-3 kinase was next examined. FIG. 8 shows sample blots and densitometry results from Western analyses probing for expression and activity of Akt (FIG. 8A), an effector of PI-3-K, or PTEN (FIG. 8B), a phosphatase that functions to dephosphorylate proteins/lipids phosphorylated by PI-3-K. Blots were probed with antibodies against total protein (Akt or PTEN) and phosphorylated protein, pAkt being active Akt and pPTEN inactive PTEN (bars represent means±SEM for the number of animals indicated by N). FIG. 8A shows the results of measuring expression of an effector of PI-3-K, Akt and its status of activation by using a phosphospecific Akt antibody (Ser 473). There was no significant difference in total Akt protein levels in aorta from WKY and SHR nor any significant difference in the pAkt protein levels.

Finally, the presence of the PI-3-K specific phosphatase PTEN was measured in the aorta of normal WKY and genetically hypertensive SHR rats. The results show that both PTEN and pPTEN were present in the aorta from SHR and WKY animals, but neither form was expressed to a different magnitude in hypertension (FIG. 8B).

These results support a specific connection between p110δ expression, but not expression of other forms of the PI-3-K p110 subunit or other factors in the PI-3-K signaling pathway, and genetically based hypertension in mammals. It is important to note that the increase in p110δ is not reflected in an increase in phosphorylation of its classical downstream substrate, Akt. Also, no difference in expression or apparent activation of a phosphatase that is specific to the functions of PI-3-K, PTEN. Collectively, these data suggest that it is p110δ itself that is the critical effector in modifying arterial tone. In summary, these collective experiments support the important of the p110δ isoform subunit of the enzyme PI-3-K in mediating arterial hypercontractility in genetic hypertension. This enzyme catalytic subunit thus represents a new target for the treatment of hypertension with specific inhibitors of p110δ activity and/or expression.

Equivalents

Those skilled in the art will recognize, or be able to ascertain using no more than routine experimentation, many equivalents of the specific embodiments of the invention described herein. Such equivalents are intended to be encompassed by the following claims.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature (see, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait, ed., 1984); Mullis et al., U.S. Pat. No. 4,683,195; Nucleic Acid Hybridization (B. D. Hames & S. J. Higgins eds. 1984); Transcription and Translation (B. D. Hames & S. J. Higgins eds. 1984); (R. I. Freshney, Alan R. Liss, Inc., 1987); Immobilized Cells and Enzymes (IRL Press, 1986); B. Perbal, A Practical Guide to Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al., eds.) Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986) (Cold Spring Harbor Laboratory Press, Cold Spring Harbor, N.Y., 1986).

1. A method of ameliorating or preventing hypertension or a condition associated with hypertension, comprising administering to an individual an amount of a phosphoinositide 3-kinase delta (PI-3-Kδ) selective inhibitor effective to ameliorate or prevent hypertension or a condition associated with hypertension and inhibit vascular p110 delta (p110δ).

2. The method according to claim 1, wherein p110δ activity is reduced.

3. The method according to claim 1, wherein p110δ expression is reduced.

4. The method according to claim 1, wherein said hypertension is essential hypertension

5. The method according to claim 1, wherein said hypertension is secondary hypertension.

6. The method according to claim 1, wherein the condition is spontaneous tone.

7. The method according to claim 5, wherein the condition is aortic spontaneous tone.

8. The method according to claim 5, wherein the condition is mesenteric resistance arterial spontaneous tone.

9. The method according to claim 1, wherein the condition is enhanced arterial contraction.

10. The method according to claim 1, wherein the condition is enhanced total peripheral resistance.

11. The method according to claim 1, wherein the inhibitor is administered in a regimen which includes administering one or more additional therapeutic compounds selected from the group consisting of ACE inhibitors, alpha-adrenoceptor agonists, alpha-adrenoceptor antagonists (alpha blockers), beta-adrenoceptor antagonists (beta blockers), angiotensin antagonists, atrial natriuretic factor, calcium channel antagonists, diuretics, dopamine receptor agonists, endopeptidase inhibitors, endothelin receptor antagonists, potassium channel agonists, renin inhibitors, serotonin antagonists, thromboxane antagonists and vasodilators.

12. The method according to claim 1, wherein the PI-3-Kδ selective inhibitor is a compound having formula (I) or pharmaceutically acceptable salts and solvates thereof: 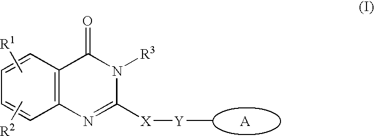
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen,
C₁₋₆alkyl, aryl, heteroaryl, halo, NHC(═O)C₁₋₃alkyleneN(R^{a})₂, NO₂, OR^{a}, CF₃,
OCF₃, N(R^{a})₂, CN, OC(═O)R^{a}, C(═O)OR^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkylene₂₋₄alkylene NR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈gheterocycloalkyl, arylOC₁₋₃alkyleneN(R^{a})₂, arylOC(═O)R^{b}, NHC(═O)C₁₋₃alkyleneC₃₋₈gheterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkylene C(═O)C₁₋₄alkyleneheteroaryl, C₁₋₄alkylenearyl optionally substituted with one or more of halo, SO₂N(R^{a})2, N(R^{a})2, C(═O)OR^{a}, NR^{a}SO₂CF₃, CN, NO₂, C(═O)R^{a}, OR^{a}, C₁₋₄alkyleneN(R^{a})2, and OC₁₋₄alkyleneN(R^{a})₂, C¹⁻⁴alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})2, C₁₋₄alkyleneOR^{a}, C₁₋₄alkyleneNR^{a}C(═O)R^{a}, C¹⁻⁴alkyleneOC¹⁻⁴CalkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃ alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

13. The method according to claim 12, wherein PI-38δ selective inhibitor is selected from the group consisting of:
2-(6-aminopurin-9-ylmethyl)-3-(2-chlorophenyl)-6,7-dimethoxy-3H-quinazolin-4-one;
2-(6-aminopurin-o-ylmethyl)-6-bromo-3-(2-chlorophenyl)-3H-quinazolin-4-one;
2-(6-aminopurin-o-ylmethyl)-3-(2-chlorophenyl)-7-fluoro-3H-quinazolin-4-one;
2-(6-aminopurin-9-ylmethyl)-6-chloro-3-(2-chlorophenyl)-3H-quinazolin-4-one;
2-(6-aminopurin-9-ylmethyl)-3-(2-chlorophenyl)-5-fluoro-3H-quinazolin-4-one;
2-(6-aminopurin-o-ylmethyl)-5-chloro-3-(2-chloro-phenyl)-3H-quinazolin-4-one;
2-(6-aminopurin-9-ylmethyl)-3-(2-chlorophenyl)-5-methyl-3H-quinazolin-4-one;
2-(6-aminopurin-9-ylmethyl)-8-chloro-3-(2-chlorophenyl)-3H-quinazolin-4-one;
2-(6-aminopurin-9-ylmethyl)-3-biphenyl-2-yl-5-chloro-3H-quinazolin-4-one;
5-chloro-2-(9H-purin-6-ylsulfanylmethyl)-3-o-tolyl-3H-quinazolin-4-one;
5-chloro-3-(2-flhorophenyl)-2-(9H-purin-6-yl-sulfanylmethyl)-3H-quinazolin-4-one;
2-(6-aminopurin-9-ylmethyl)-5-chloro-3-(2-fluorophenyl)-3H-quinazolin-4-one;
3-biphenyl-2-yl-5-chloro-2-(9H-purin-6-ylsulfanylmethyl)-3H-quinazolin-4-one;
5-chloro-3-(2-methoxyphenyl)-2-(9H-purin-6-yl-sulfanylmethyl)-3H-quinazolin-4-one;
3-(2-chlorophenyl)-5-fluoro-2-(9H-purin-6-yl-sulfanylmethyl)-3H-quinazolin-4-one;
3-(2-chlorophenyl)-6,7-dimethoxy-2-(9H-purin-6-yl-sulfanylmethyl)-3H-quinazolin-4-one;
6-bromo-3-(2-chlorophenyl)-2-(9H-purin-6-yl-sulfanylmethyl)-3H-quinazolin-4-one;
3-(2-chlorophenyl)-8-trifluoromethyl-2-(9H-purin-6-ylsulfanylmethyl)-3H-quinazolin-4-one;
3-(2-chlorophenyl)-2-(9H-purin-6-ylsulfanylmethyl)-3H-benzo[g]quinazolin-4-one;
6-chloro-3-(2-chlorophenyl)-2-(9H-purin-6-yl-sulfanylmethyl)-3H-quinazolin-4-one;
8-chloro-3-(2-chlorophenyl)-2-(9H-purin-6-yl-sulfanylmethyl)-3H-quinazolin-4-one;
3-(2-chlorophenyl)-7-fluoro-2-(9H-purin-6-yl-sulfanylmethyl)-3H-quinazolin-4-one;
3-(2-chlorophenyl)-7-nitro-2-(9H-purin-6-yl-sulfanylmethyl)-3H-quinazolin-4-one;
3-(2-chlorophenyl)-6-hydroxy-2-(9H-purin-6-yl-sulfanylmethyl)-3H-quinazolin-4-one;
5-chloro-3-(2-chlorophenyl)-2-(9H-purin-6-yl-sulfanylmethyl)-3H-quinazolin-4-one;
3-(2-chlorophenyl)-5-methyl-2-(9H-purin-6-yl-sulfanylmethyl)-3H-quinazolin-4-one;
3-(2-chlorophenyl)-6,7-difluoro-2-(9H-purin-6-yl-sulfanylmethyl)-3H-quinazolin-4-one;
3-(2-chlorophenyl)-6-fluoro-2-(9H-purin-6-yl-sulfanylmethyl)-3H-quinazolin-4-one;
2-(6-aminopurin-9-ylmethyl)-3-(2-isopropylphenyl)-5-methyl-3H-quinazolin-4-one;
2-(6-aminopurin-9-ylmethyl)-5-methyl-3-o-tolyl-3H-quinazolin-4-one;
3-(2-fluorophenyl)-5-methyl-2-(9H-purin-6-yl-sulfanylmethyl)-3H-quinazolin-4-one;
2-(6-aminopurin-9-ylmethyl)-5-chloro-3-o-tolyl-3H-quinazolin-4-one;
2-(6-aminopurin-9-ylmethyl)-5-chloro-3-(2-methoxy-phenyl)-3H-quinazolin-4-one;
2-(2-amino-9H-purin-6-ylsulfanylmethyl)-3-cyclopropyl-5-methyl-3H=quinazolin-4-one;
3-cyclopropylmethyl-5-methyl-2-(9H-purin-6-ylsulfanylmethyl)-3H-quinazolin-4-one;
2-(6-aminopurin-9-ylmethyl)-3-cyclopropylmethyl-5-methyl-3H-quinazolin-4-one;
2-(2-amino-9H-purin-6-ylsulfanylmethyl)-3-cyclopropylmethyl-5-methyl-3H-quinazolin-4-one;
5-methyl-3-phenethyl-2-(9H-purin-6-ylsulfanylmethyl)-3H-quinazolin-4-one;
2-(2-amino-9H-purin-6-ylsulfanylmethyl)-5-methyl-3-phenethyl-3H-quinazolin-4-one;
3-cyclopentyl-5-methyl-2-(9H-purin-6-ylsulfanylmethyl)-3H-quinazolin-4-one;
2-(6-aminopurin-9-ylmethyl)-3-cyclopentyl-5-methyl-3H-quinazolin-4-one;
3-(2-chloropyridin-3-yl)-5-methyl-2-(9H-purin-6-ylsulfanylmethyl)-3H-quinazolin-4-one;
2-(6-aminopurin-9-ylmethyl)-3-(2-chloropyridin-3-yl)-5-methyl-3H-quinazolin-4-one;
3-methyl-4-[5-methyl-4-oxo-2-(9H-purin-6-ylsulfanylmethyl)-4H-quinazolin-3-yl]-benzoic acid;
3-cyclopropyl-5-methyl-2-(9H-purin-6-ylsulfanylmethyl)-3H-quinazolin-4-one;
2-(6-aminopurin-9-ylmethyl)-3-cyclopropyl-5-methyl-3H-quinazolin-4-one;
5-methyl-3-(4-nitrobenzyl)-2-(9H-purin-6-ylsulfanylmethyl)-3H-quinazolin-4-one;
3-cyclohexyl-5-methyl-2- (9H-purin-6-ylsulfanyl methyl)-3H-quinazolin-4-one;
2-(6-aminopurin-9-ylmethyl)-3-cyclohexyl-5-methyl-3H-quinazolin-4-one;
2-(2-amino-9H-purin-6-ylsulfanylmethyl)-3-cyclo-hexyl-5-methyl-3H-quinazolin-4-one;
5-methyl-3-(E-2-phenylcyclopropyl)-2-(9H-purin-6-ylsulfanylmethyl)-3H-quinazolin-4-one;
3-(2-chlorophenyl)-5-fluoro-2-[(9H-purin-6-ylamino)methyl]-3H-quinazolin-4-one;
2-[(2-amino-9H-purin-6-ylamino)methyl]-3-(2-chlorophenyl)-5-fluoro-3H-quinazolin-4-one;
5-methyl-2-[(9H-purin-6-ylamino)methyl]-3-o-tolyl-3H-quinazolin-4-one;
2-[(2-amino-9H-purin-6-ylamino)methyl]-5-methyl-3-o-tolyl-3H-quinazolin-4-one;
2-[(2-fluoro-9H-purin-6-ylamino)methyl]-5-methyl-3-o-tolyl-3H-quinazolin-4-one;
(2-chlorophenyl)-dimethylamino-(9H-purin-6-ylsulfanylmethyl)-3H-quinazolin-4-one;
5-(2-benzyloxyethoxy)-3-(2-chlorophenyl)-2-(9H-purin-6-ylsulfanylmethyl)-3H-quinazolin-4-one;
6-aminopurine-9-carboxylic acid 3-(2-chlorophenyl)-5-fluoro-4-oxo-3,4-dihydroquinazolin-2-ylmethyl ester;
N-[3-(2-chlorophenyl)-5-fluoro-4-oxo-3,4-dihydro-quinazolin-2-ylmethyl]-2-(9H-purin-6-ylsulfanyl)-acetamide;
2-[1-(2-fluoro-9H-purin-6-ylamino)ethyl]-5-methyl-3-o-tolyl-3H-quinazolin-4-one;
5-methyl-2-[1-(9H-purin-6-ylamino)ethyl]-3-o-tolyl-3H-quinazolin-4-one;
2-(6-dimethylanopurin-9-ylmethyl)-5-methyl-3-o-tolyl-3H-quinazolin-4-one;
5-methyl-2-(2-methyl-6-oxo-1,6-dihydro-purin-7-ylmethyl)-3-o-tolyl-3H-quinazolin-4-one;
5-methyl-2-(2-methyl-6-oxo-1,6-dihydro-purin-9-ylmethyl)-3-o-tolyl-3H-quinazolin-4-one;
2-(amino-dimethylaminopurin-9-ylmethyl)-5-methyl-3-o-tolyl-3H-quinazolin-4-one;
2-(2-amino-9H-purin-6-ylsulfanylmethyl)-5-methyl-3-o-tolyl-3H-quinazolin-4-one;
2-(4-amino-1,3;5-triazin-2-ylsulfanylmethyl)-5-methyl-3-o-tolyl-3H-quinazolin-4-one;
5-methyl-2-(7-methyl-7H-purin-6-ylsulfanylmethyl)-3-o-tolyl-3H-quinazolin-4-one;
5-methyl-2-(2-oxo-1,2-dihydro-pyrimidin-4-ylsulfanylmethyl)-3-o-tolyl-3H-quinazolin-4-one;
5-methyl-2-purin-7-ylmethyl-3-o-tolyl-3H-quinazolin-4-one;
5-methyl-2-purin-9-ylmethyl-3-o-tolyl-3H-quinazolin-4-one;
5-methyl-2-(9-methyl-9H-purin-6-ylsulfanylmethyl)-3-o-tolyl-3H-quinazolin-4-one;
2-(2,6-Diamino-pyrimidin-4-ylsulfanylmethyl)-5-methyl-3-o-tolyl-3H-quinazolin-4-one;
5-methyl-2-(5-methyl-[1,2,4]triazolo[1,5-a]pyrimidin-7-ylsulfanylmethyl)-3-o-tolyl-3H-quinazolin-4-one;
5-methyl-2-(2-methylsulfanyl-9H-purin-6-ylsulfanylmethyl)-3-o-tolyl-3H-quinazolin-4-one;
2-(2-hydroxy-9H-purin-6-ylsulfanylmethyl)-5-methyl-3-o-tolyl-3H-quinazolin-4-one;
5-methyl-2-(1-methyl-1H-imidazol-2-ylsulfanylmethyl)-3-o-tolyl-3H-quinazolin-4-one;
5-methyl-3-o-tolyl-2-(1H-[1,2,4]triazol-3-ylsulfanylmethyl)-3H-quinazolin-4-one;
2-(2-amino-6-chloro-purin-9-ylmethyl)-5-methyl-3-o-tolyl-3H-quinazolin-4-one;
2-(6-aminopurin-7-ylmethyl)-5-methyl-3-o-tolyl-3H-quinazolin-4-one;
2-(7-amino-1,2,3-triazolo [4,5-d]pyrimidin-3-yl-methyl)-5-methyl-3-o-tolyl-3H-quinazolin-4-one;
2-(7-amino-1,2,3-triazolo[4,5-d]pyrimidin-1-yl-methyl)-5-methyl-3-o-tolyl-3H-quinazolin-4-one;
2-(6-amino-9H-purin-2-ylsulfanylmethyl)-5-methyl-3-o-tolyl-3H-quinazolin-4-one;
2-(2-amino-6-ethylamino-pyrimidin-4-ylsulfanylmethyl)-5-methyl-3-o-tolyl-3H-quinazolin-4-one;
2-(3-amino-5-methylsulfanyl-1,2,4-triazol-1-yl-methyl)-5-methyl-3-o-tolyl-3H-quinazolin-4-one;
2-(5-amino-3-methylsulfanyl-1,2,4-triazol-1-ylmethyl)-5-methyl-3-o-tolyl-3H-quinazolin-4-one;
5-methyl-2-(6-methylaminopurin-9-ylmethyl)-3-o-tolyl-3H-quinazolin-4-one;
2-(6-benzylaminopurin-9-ylmethyl)-5-methyl-3-o-tolyl-3H-quinazolin-4-one;
2-(2,6-diaminopurin-9-ylmethyl)-5-methyl-3-o-tolyl-3H-quinazolin-4-one;
5-methyl-2-(9H-purin-6-ylsulfanylmethyl)-3-o-tolyl-3H-quinazolin-4-one;
3-isobutyl-5-methyl-2-(9H-purin-6-ylsulfanylmethyl)-3H-quinazolin-4-one;
N-{2-[5-Methyl-4-oxo-2-(9H-purin-6-ylsulfanylmethyl)-4H-quinazolin-3-yl]-phenyl}-acetamide;
5-methyl-3-(E-2-methyl-cyclohexyl)-2-(9H-purin-6-ylsulfanylmethyl)-3H-quinazolin-4-one;
2-[5-methyl-4-oxo-2-(9H-purin-6-ylsulfanylmethyl)-4H-quinazolin-3-yl]-benzoic acid;
3-{2-[(2-dimethyl aminoethyl)methylamino]phenyl}-5-methyl-2-(9H-purin-6-ylsulfanylmethyl)-3H-quinazolin-4-one;
3-(2-chlorophenyl)-5-methoxy-2-(9H-purin-6-ylsulfanylmethyl)-3H-quinazolin-4-one;
3-(2-chlorophenyl)-5-(2-morpholin-4-yl-ethylamino)-2-(9H-purin-6-ylsulfanylmethyl)-3H-quinazolin-4-one;
3-benzyl-5-methoxy-2-(9H-purin-6-ylsulfanylmethyl)-3H-quinazolin-4-one;
2-(6-aminopurin-9-ylmethyl)-3-(2-benzyloxyphenyl)-5-methyl-3H-quinazolin-4-one;
2-(6-aminopurin-9-ylmethyl)-3-(2-hydroxyphenyl)-5-methyl-3H-quinazolin-4-one;
2-(1-(2-amino-9H-purin-6-ylamino)ethyl)-5-methyl-3-o-tolyl-3H-quinazolin-4-one;
5-methyl-2-[ ]-(9H-purin-6-ylamino)propyl]-3-o-tolyl-3H-quinazolin-4-one;
2-(1-(2-fluoro-9H-purin-6-ylamino)propyl)-5-methyl-3-o-tolyl-3H-quinazolin-4-one;
2-(1-(2-amino-9H-purin-6-ylamino)propyl)-5-methyl-3-o-tolyl-3H-quinazolin-4-one;
2-(2-benzyloxy-1-(9H-purin-6-ylamino)ethyl)-5-methyl-3-o-tolyl-3H-quinazolin-4-one;
2-(6-aminopurin-9-ylmethyl)-5-methyl-3-{2-(2-(1-methylpyrrolidin-2-yl)-ethoxy)-phenyl}-3H-quinazolin-4-one;
2-(6-aminopurin-9-ylmethyl)-3-(2-(3-dimethylamino-propoxy)-phenyl)-5-methyl-3H-quinazolin-4-one;
2-(6-aminopurin-9-ylmethyl)-5-methyl-3-(2-prop-2-ynyloxyphenyl)-3H-quinazolin-4-one; and
2-{2-(1-(6-aminopurin-9-ylmethyl)-5-methyl-4-oxo-4H-quinazolin-3-yl]-phenoxy}-acetamide, and
pharmaceutically acceptable salts and solvates thereof.

14. A method of treating hypertension or a condition associated with hypertension, comprising:
identifying a subject with hypertension or a condition associated with hypertension; and
administering to the subject an amount of a phosphoinositide 3-kinase delta (PI3Kδ) selective inhibitor effective to treat the hypertension or the condition associated with hypertension,
thereby treating hypertension or a condition associated with hypertension in the subject.

15. The method of claim 14, wherein the subject is a human subject.

16. The method of claim 14, wherein the subject is a mammal.

17. The method of claim 16, wherein the subject is a rat or a mouse.

18. The method of claim 17, wherein the rat or mouse has genetically-based hypertension.

19. The method of claim 17, wherein the subject has deoxycorticosterone acetate (DOCA)-salt induced hypertension.

20. The method according to claim 14, wherein the hypertension is essential hypertension.

21. The method according to claim 14, wherein the hypertension is secondary hypertension.

22. The method according to claim 14, wherein the condition is spontaneous tone.

23. The method according to claim 14, wherein the condition is aortic spontaneous tone.

24. The method according to claim 14, wherein the condition is mesenteric resistance arterial spontaneous tone.

25. The method according to claim 14, wherein the condition is enhanced arterial contraction.

26. The method according to claim 14, wherein the condition is enhanced total peripheral resistance.

27. The method according to claim 14, wherein the inhibitor is administered in a regimen which includes administering one or more additional therapeutic compounds selected from the group consisting of ACE inhibitors, alpha-adrenoceptor agonists, alpha-adrenoceptor antagonists (alpha blockers), beta-adrenoceptor antagonists (beta blockers), angiotensin antagonists, atrial natriuretic factor, calcium channel antagonists, diuretics, dopamine receptor agonists, endopeptidase inhibitors, endothelin receptor antagonists, potassium channel agonists, renin inhibitors, serotonin antagonists, thromboxane antagonists, and vasodilators.

28. The method according to claim 14, wherein p110δ activity is reduced.

29. The method according to claim 14, wherein p110δ expression is reduced.

30. The method according to claim 28, wherein the PI-3-Kδ selective inhibitor is a compound having formula (I) or pharmaceutically acceptable salts and solvates thereof: 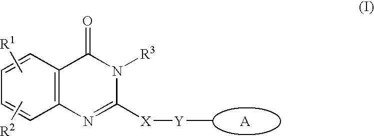
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen,
C₁₋₆alkyl, aryl, heteroaryl, halo, NHC(═O)C₁₋₃alkyleneN(R^{a})₂, NO₂, OR^{a}, CF₃,
OCF₃, N(R^{a})₂, CN, OC(═O)R^{a}, C(═O)OR^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C(═O)NR^{a}SO₂R^{a}, C₁₋₄alkyleneN(R^{a})2, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkylene₂₋₄alkylene NR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R)₂, C(═O)N(R^{a})₂, NHC(═O)C₁₋₃alkylenearyl, C₃₋₈cycloalkyl, C₃₋₈gheterocycloalkyl, arylOC₁₋₃alkyleneN(R^{a})₂, arylOC(═O)R^{b}, NHC(═O)C₁₋₃alkyleneC₃₋₈gheterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneORₐ, C(═O)NR^{a}C₁₋₄alkylene C(═O)C₁₋₄alkyleneheteroaryl, C₁₋₄alkylenearyl optionally substituted with one or more of halo, SO₂N(R^{a})2, N(R^{a})2, C(═O)OR^{a}, NR^{a}SO₂CF₃, CN, NO₂, C(═O)R^{a}, OR^{a}, C₁₋₄alkyleneN(R^{a})2, and OC₁₋₄alkyleneN(R^{a})₂, C¹⁻⁴alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})2, C₁₋₄alkyleneOR^{a}, C¹⁻⁴alkyleneNR^{a}C(═O)R^{a}, C¹⁻⁴alkyleneOC¹⁻⁴alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃ alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

31. The method according to claim 28, wherein PI-3-Kδ selective inhibitor is selected from the group consisting of:
2-(6-aminopurin-9-ylmethyl)-3-(2-chlorophenyl)-6,7-dimethoxy-3H-quinazolin-4-one;
2-(6-aminopurin-o-ylmethyl)-6-bromo-3-(2-chlorophenyl)-3H-quinazolin-4-one;
2-(6-aminopurin-o-ylmethyl)-3-(2-chlorophenyl)-7-fluoro-3H-quinazolin-4-one;
2-(6-aminopurin-9-ylmethyl)-6-chloro-3-(2-chlorophenyl)-3H-quinazolin-4-one;
2-(6-aminopurin-9-ylmethyl)-3-(2-chlorophenyl)-5-fluoro-3H-quinazolin-4-one;
2-(6-aminopurin-o-ylmethyl)-5-chloro-3-(2-chloro-phenyl)-3H-quinazolin-4-one;
2-(6-aminopurin-9-ylmethyl)-3-(2-chlorophenyl)-5-methyl-3H-quinazolin-4-one;
2-(6-aminopurin-9-ylmethyl)-8-chloro-3-(2-chlorophenyl)-3H-quinazolin-4-one;
2-(6-aminopurin-9-ylmethyl)-3-biphenyl-2-yl-5-chloro-3H-quinazolin-4-one;
5-chloro-2-(9H-purin-6-ylsulfanylmethyl)-3-o-tolyl-3H-quinazolin-4-one;
5-chloro-3-(2-flhorophenyl)-2-(9H-purin-6-yl-sulfanylmethyl)-3H-quinazolin-4-one;
2-(6-aminopurin-9-ylmethyl)-5-chloro-3-(2-fluorophenyl)-3H-quinazolin-4-one;
3-biphenyl-2-yl-5-chloro-2-(9H-purin-6-ylsulfanylmethyl)-3H-quinazolin-4-one;
5-chloro-3-(2-methoxyphenyl)-2-(9H-purin-6-yl-sulfanylmethyl)-3H-quinazolin-4-one;
3-(2-chlorophenyl)-5-fluoro-2-(9H-purin-6-yl-sulfanylmethyl)-3H-quinazolin-4-one;
3-(2-chlorophenyl)-6,7-dimethoxy-2-(9H-purin-6-yl-sulfanylmethyl)-3H-quinazolin-4-one;
6-bromo-3-(2-chlorophenyl)-2-(9H-purin-6-yl-sulfanylmethyl)-3H-quinazolin-4-one;
3-(2-chlorophenyl)-8-trifluoromethyl-2-(9H-purin-6-ylsulfanylmethyl)-3H-quinazolin-4-one;
3-(2-chlorophenyl)-2-(9H-purin-6-ylsulfanylmethyl)-3H-benzo[g]quinazolin-4-one;
6-chloro-3-(2-chlorophenyl)-2-(9H-purin-6-yl-sulfanylmethyl)-3H-quinazolin-4-one;
8-chloro-3-(2-chlorophenyl)-2-(9H-purin-6-yl-sulfanylmethyl)-3H-quinazolin-4-one;
3-(2-chlorophenyl)-7-fluoro-2-(9H-purin-6-yl-sulfanylmethyl)-3H-quinazolin-4-one;
3-(2-chlorophenyl)-7-nitro-2-(9H-purin-6-yl-sulfanylmethyl)-3H-quinazolin-4-one;
3-(2-chlorophenyl)-6-hydroxy-2-(9H-purin-6-yl-sulfanylmethyl)-3H-quinazolin-4-one;
5-chloro-3-(2-chlorophenyl)-2-(9H-purin-6-yl-sulfanylmethyl)-3H-quinazolin-4-one;
3-(2-chlorophenyl)-5-methyl-2-(9H-purin-6-yl-sulfanylmethyl)-3H-quinazolin-4-one;
3-(2-chlorophenyl)-6,7-difluoro-2-(9H-purin-6-yl-sulfanylmethyl)-3H-quinazolin-4-one;
3-(2-chlorophenyl)-6-fluoro-2-(9H-purin-6-yl-sulfanylmethyl)-3H-quinazolin-4-one;
2-(6-aminopurin-9-ylmethyl)-3-(2-isopropylphenyl)-5-methyl-3H-quinazolin-4-one;
2-(6-aminopurin-9-ylmethyl)-5-methyl-3-o-tolyl-3H-quinazolin-4-one;
3-(2-fluorophenyl)-5-methyl-2-(9H-purin-6-yl-sulfanylmethyl)-3H-quinazolin-4-one;
2-(6-aminopurin-9-ylmethyl)-5-chloro-3-o-tolyl-3H-quinazolin-4-one;
2-(6-aminopurin-9-ylmethyl)-5-chloro-3-(2-methoxy-phenyl)-3H-quinazolin-4-one;
2-(2-amino-9H-purin-6-ylsulfanylmethyl)-3-cyclopropyl-5-methyl-3H=quinazolin-4-one;
3-cyclopropylmethyl-5-methyl-2-(9H-purin-6-ylsulfanylmethyl)-3H-quinazolin-4-one;
2-(6-aminopurin-9-ylmethyl)-3-cyclopropylmethyl-5-methyl-3H-quinazolin-4-one;
2-(2-amino-9H-purin-6-ylsulfanylmethyl)-3-cyclopropylmethyl-5-methyl-3Hquinazolin-4-one;
5-methyl-3-phenethyl-2-(9H-purin-6-ylsulfanylmethyl)-3H-quinazolin-4-one;
2-(2-amino-9H-purin-6-ylsulfanylmethyl)-5-methyl-3-phenethyl-3H-quinazolin-4-one;
3-cyclopentyl-5-methyl-2-(9H-purin-6-ylsulfanylmethyl)-3H-quinazolin-4-one;
2-(6-aminopurin-9-ylmethyl)-3-cyclopentyl-5-methyl-3H-quinazolin-4-one;
3-(2-chloropyridin-3-yl)-5-methyl-2-(9H-purin-6-ylsulfanylmethyl)-3H-quinazolin-4-one;
2-(6-aminopurin-9-ylmethyl)-3-(2-chloropyridin-3-yl)-5-methyl-3H-quinazolin-4-one;
3-methyl-4-[5-methyl-4-oxo-2-(9H-purin-6-ylsulfanylmethyl)-4H-quinazolin-3-yl]-benzoic acid;
3-cyclopropyl-5-methyl-2-(9H-purin-6-ylsulfanylmethyl)-3H-quinazolin-4-one;
2-(6-aminopurin-9-ylmethyl)-3-cyclopropyl-5-methyl-3H-quinazolin-4-one;
5-methyl-3-(4-nitrobenzyl)-2-(9H-purin-6-ylsulfanylmethyl)-3H-quinazolin-4-one;
3-cyclohexyl-5-methyl-2-(9H-purin-6-ylsulfanylmethyl)-3H-quinazolin-4-one;
2-(6-aminopurin-9-ylmethyl)-3-cyclohexyl-5-methyl-3H-quinazolin-4-one;
2-(2-amino-9H-purin-6-ylsulfanylmethyl)-3-cyclo-hexyl-5-methyl-3H-quinazolin-4-one;
5-methyl-3-(E-2-phenylcyclopropyl)-2-(9H-purin-6-ylsulfanylmethyl)-3H-quinazolin-4-one;
3-(2-chlorophenyl)-5-fluoro-2-[(9H-purin-6-ylamino)methyl]-3H-quinazolin-4-one;
2-[(2-amino-9H-purin-6-ylamino)methyl]-3-(2-chlorophenyl)-5-fluoro-3H-quinazolin-4-one;
5-methyl-2-[(9H-purin-6-ylamino)methyl]-3-o-tolyl-3H-quinazolin-4-one;
2-[(2-amino-9H-purin-6-ylamino)methyl]-5-methyl-3-o-tolyl-3H-quinazolin-4-one;
2-[(2-fluoro-9H-purin-6-ylamino)methyl]-5-methyl-3-o-tolyl-3H-quinazolin-4-one;
(2-chlorophenyl)-dimethylamino-(9H-purin-6-ylsulfanylmethyl)-3H-quinazolin-4-one;
5-(2-benzyloxyethoxy)-3-(2-chlorophenyl)-2-(9H-purin-6-ylsulfanylmethyl)-3H-quinazolin-4-one;
6-aminopurine-9-carboxylic acid 3-(2-chlorophenyl)-5-fluoro-4-oxo-3,4-dihydroquinazolin-2-ylmethyl ester;
N-[3-(2-chlorophenyl)-5-fluoro-4-oxo-3,4-dihydro-quinazolin-2-ylmethyl]-2-(9H-purin-6-ylsulfanyl)-acetamide;
2-[1-(2-fluoro-9H-purin-6-ylamino)ethyl]-5-methyl-3-o-tolyl-3H-quinazolin-4-one;
5-methyl-2-[1-(9H-purin-6-ylamino)ethyl]-3-o-tolyl-3H-quinazolin-4-one;
2-(6-dimethylaminopurin-9-ylmethyl)-5-methyl-3-o-tolyl-3H-quinazolin-4-one;
5-methyl-2-(2-methyl-6-oxo-1,6-dihydro-purin-7-ylmethyl)-3-o-tolyl-3H-quinazolin-4-one;
5-methyl-2-(2-methyl-6-oxo-1,6-dihydro-purin-9-ylmethyl)-3-o-tolyl-3H-quinazolin-4-one;
2-(amino-dimethylaminopurin-9-ylmethyl)-5-methyl-3-o-tolyl-3H-quinazolin-4-one;
2-(2-amino-9H-purin-6-ylsulfanylmethyl)-5-methyl-3-o-tolyl-3H-quinazolin-4-one;
2-(4-amino-1,3;5-triazin-2-ylsulfanylmethyl)-5-methyl-3-o-tolyl-3H-quinazolin-4-one;
5-methyl-2-(7-methyl-7H-purin-6-ylsulfanylmethyl)-3-o-tolyl-3H-quinazolin-4-one;
5-methyl-2-(2-oxo-1,2-dihydro-pyrimidin-4-ylsulfanylmethyl)-3-o-tolyl-3H-quinazolin-4-one;
5-methyl-2-purin-7-ylmethyl-3-o-tolyl-3H-quinazolin-4-one;
5-methyl-2-purin-9-ylmethyl-3-o-tolyl-3H-quinazolin-4-one;
5-methyl-2-(9-methyl-9H-purin-6-ylsulfanylmethyl)-3-o-tolyl-3H-quinazolin-4-one;
2-(2,6-Diamino-pyrimidin-4-ylsulfanylmethyl)-5-methyl-3-o-tolyl-3H-quinazolin-4-one;
5-methyl-2-(5-methyl-[1,2,4]triazolo[1,5-a]pyrimidin-7-ylsulfanylmethyl)-3-o-tolyl-3H-quinazolin-4-one;
5-methyl-2-(2-methylsulfanyl-9H-purin-6-ylsulfanylmethyl)-3-o-tolyl-3H-quinazolin-4-one;
2-(2-hydroxy-9H-purin-6-ylsulfanylmethyl)-5-methyl-3-o-tolyl-3H-quinazolin-4-one;
5-methyl-2-(1-methyl-1H-imidazol-2-ylsulfanylmethyl)-3-o-tolyl-3H-quinazolin-4-one;
5-methyl-3-o-tolyl-2-(1H-[1,2,4]triazol-3-ylsulfanylmethyl)-3H-quinazolin-4-one;
2-(2-amino-6-chloro-purin-9-ylmethyl)-5-methyl-3-o-tolyl-3H-quinazolin-4-one;
2-(6-aminopurin-7-ylmethyl)-5-methyl-3-o-tolyl-3H-quinazolin-4-one;
2-(7-amino-1,2,3-triazolo[4,5-d]pyrimidin-3-yl-methyl)-5-methyl-3-o-tolyl-3H-quinazolin-4-one;
2-(7-amino-1,2,3-triazolo[4,5-d]pyrimidin-1-yl-methyl)-5-methyl-3-o-tolyl-3H-quinazolin-4-one;
2-(6-amino-9H-purin-2-ylsulfanylmethyl)-5-methyl-3-o-tolyl-3H-quinazolin-4-one;
2-(2-amino-6-ethylamino-pyrimidin-4-ylsulfanylmethyl)-5-methyl-3-o-tolyl-3H-quinazolin-4-one;
2-(3-amino-5-methylsulfanyl-1,2,4-triazol-1-yl-methyl)-5-methyl-3-o-tolyl-3H-quinazolin-4-one;
2-(5-amino-3-methylsulfanyl-1,2,4-triazol-1-ylmethyl)-5-methyl-3-o-tolyl-3H-quinazolin-4-one;
5-methyl-2-(6-methylaminopurin-9-ylmethyl)-3-o-tolyl-3H-quinazolin-4-one;
2-(6-benzylaminopurin-9-ylmethyl)-5-methyl-3-o-tolyl-3H-quinazolin-4-one;
2-(2,6-diaminopurin-9-ylmethyl)-5-methyl-3-o-tolyl-3H-quinazolin-4-one;
5-methyl-2-(9H-purin-6-ylsulfanylmethyl)-3-o-tolyl-3H-quinazolin-4-one;
3-isobutyl-5-methyl-2-(9H-purin-6-ylsulfanylmethyl)-3H-quinazolin-4-one;
N-{2-[5-Methyl-4-oxo-2-(9H-purin-6-ylsulfanylmethyl)-4H-quinazolin-3-yl]-phenyl}-acetamiide;
5-methyl-3-(E-2-methyl-cyclohexyl)-2-(9H-purin-6-ylsulfanylmethyl)-3H-quinazolin-4-one;
2-[5-methyl-4-oxo-2-(9H-purin-6-ylsulfanylmethyl)-4H-quinazolin-3-yl]-benzoic acid;
3-{2-[(2-dimethyl aminoethyl)methylamino]phenyl}-5-methyl-2-(9H-purin-6-ylsulfanylmethyl)-3H-quinazolin-4-one;
3-(2-chlorophenyl)-5-methoxy-2-(9H-purin-6-ylsulfanylmethyl)-3H-quinazolin-4-one;
3-(2-chlorophenyl)-5-(2-morpholin-4-yl-ethylamino)-2-(9H-purin-6-ylsulfanylmethyl)-3H-quinazolin-4-one;
3-benzyl-5-methoxy-2-(9H-purin-6-ylsulfanylmethyl)-3H-quinazolin-4-one;
2-(6-aminopurin-9-ylmethyl)-3-(2-benzyloxyphenyl)-5-methyl-3H-quinazolin-4-one;
2-(6-aminopurin-9-ylmethyl)-3-(2-hydroxyphenyl)-5-methyl-3H-quinazolin-4-one;
2-(1-(2-amino-9H-purin-6-ylamino)ethyl)-5-methyl-3-o-tolyl-3H-quinazolin-4-one;
5-methyl-2-[ ]-(9H-purin-6-ylamino)propyl]-3-o-tolyl-3H-quinazolin-4-one;
2-(1-(2-fluoro-9H-purin-6-ylamino)propyl)-5-methyl-3-o-tolyl-3H-quinazolin-4-one;
2-(1-(2-amino-9H-purin-6-ylamino)propyl)-5-methyl-3-o-tolyl-3H-quinazolin-4-one;
2-(2-benzyloxy-1-(9H-purin-6-ylamino)ethyl)-5-methyl-3-o-tolyl-3H-quinazolin-4-one;
2-(6-aminopurin-9-ylmethyl)-5-methyl-3-{2-(2-(1-methylpyrrolidin-2-yl)-ethoxy)-phenyl}-3H-quinazolin-4-one;
2-(6-aminopurin-9-ylmethyl)-3-(2-(3-dimethylamino-propoxy)-phenyl)-5-methyl-3H-quinazolin-4-one;
2-(6-aminopurin-9-ylmethyl)-5-methyl-3-(2-prop-2-ynyloxyphenyl)-3H-quinazolin-4-one; and
2-{2-(1-(6-aminopurin-9-ylmethyl)-5-methyl-4-oxo-4H-quinazolin-3-yl]-phenoxy}-acetamide, and
pharmaceutically acceptable salts and solvates thereof.

32. The method of claim 28, wherein the PI-3-Kδ selective inhibitor is an aptamer

33. The method of claim 29, wherein the PI-3-Kδ selective inhibitor is selected from the group consisting of a ribozyme, an antisense oligonucleotide, and a siRNA.

34. The method of claim 13, wherein the wherein the PI-38δ selective inhibitor is 2-(6-Amino-purin-9-ylmethyl)-5-methyl-3-o-tolyl-3H-quinazolin-4-one.

35. The method of claim 31, wherein the wherein the PI-38δ selective inhibitor is 2-(6-Amino-purin-9-ylmethyl)-5-methyl-3-o-tolyl-3H-quinazolin-4-one.

36. A method of ameliorating or preventing hypertension or a condition associated with hypertension, comprising administering to an individual an amount of a phosphoinositide 3-kinase delta (PI-3-Kδ) selective inhibitor having the structure 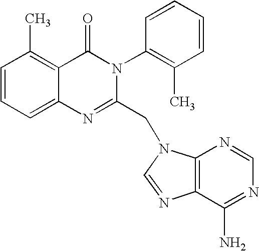
in an amount effective to ameliorate or prevent hypertension, or a condition associated with hypertension, and inhibit vascular p110 delta (p110δ).

37. A method of treating hypertension or a condition associated with hypertension, comprising:
identifying a subject with hypertension or a condition associated with hypertension; and
administering to the subject an amount of a phosphoinositide 3-kinase delta (PI-3-Kδ) selective inhibitor having the structure 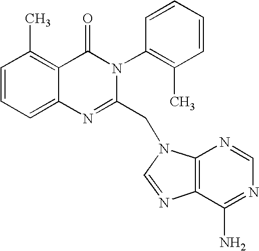
in an amount effective to ameliorate or prevent hypertension, or a condition associated with hypertension, and inhibit vascular p110 delta (p110δ),
thereby treating hypertension or a condition associated with hypertension in the subject.


SEQUENCE LISTING (2 sequences)

SEQ ID NO: 1 - protein, 1044 aa, Homo sapiens
MPPGVDCPMEFWTKEENQSVVVDFLLPTGVYLNFPVSRNANLSTIKQLLWHRAQYEPLFHMLSGPEAYVFTCINQTAEQQELEDEQRRLCDVQPFLPVLRLVAREGDRVKKLINSQISLLIGKGLHEFDSLCDPEVNDFRAKMCQFCEEAAARRQQLGWEAWLQYSFPLQLEPSAQTWGPGTLRLPNRALLVNVKFEGSEESFTFQVSTKDVPLALMACALRKKATVFRQPLVEQPEDYTLQVNGRHEYLYGNYPLCQFQYICSCLHSGLTPHLTMVHSSSILAMRDEQSNPAPQVQKPRAKPPPIPAKKPSSVSLWSLEQPFRIELIQGSKVNADERMKLVVQAGLFHGNEMLCKTVSSSEVSVCSEPVWKQRLEFDINICDLPRMARLCFALYAVIEKAKKARSTKKKSKKADCPIAWANLMLFDYKDQLKTGERCLYMWPSVPDEKGELLNPTGTVRSNPNTDSAAALLICLPEVAPHPVYYPALEKILELGRHSECVHVTEEEQLQLREILERRGSGELYEHEKDLVWKLRHEVQEHFPEALARLLLVTKWNKHEDVAQMLYLLCSWPELPVLSALELLDFSFPDCHVGSFAIKSLRKLTDDELFQYLLQLVQVLKYESYLDCELTKFLLDRALANRKIGHFLFWHLRSEMHVPSVALRFGLILEAYCRGSTHHMKVLMKQGEALSKLKALNDFVKLSSQKTPKPQTKELMHLCMRQEAYLEALSHLQSPLDPSTLLAEVCVEQCTFMDSKMKPLWIMYSNEEAGSGGSVGIIFKNGDDLRQDMLTLQMIQLMDVLWKQEGLDLRMTPYGCLPTGDRTGLIEVVLRSDTIANIQLNKSNMAATAAFNKDALLNWLKSKNPGEALDRAIEEFTLSCAGYCVATYVLGIGDRHSDNIMIRESGQLFHIDFGHFLGNFKTKFGINRERVPFILTYDFVHVIQQGKTNNSEKFERFRGYCERAYTILRRHGLLFLHLFALMRAAGLPELSCSKDIQYLKDSLALGKTEEEALKHFRVKFNEALRESWKTKVNWLAHNVSKDNRQ

SEQ ID NO: 2 - DNA, 5220 bp, Homo sapiens
cagtcgctccgagcggccgcgagcagagccgcccagccctgtcagctgcgccgggacgataaggagtcaggccagggcgggatgacactcattgattctaaagcatctttaatctgccaggcggagggggctttgctggtctttcttggactattccagagaggacaactgtcatctgggaagtaacaacgcaggatgccccctggggtggactgccccatggaattctggaccaaggaggagaatcagagcgttgtggttgacttcctgctgcccacaggggtctacctgaacttccctgtgtcccgcaatgccaacctcagcaccatcaagcagctgctgtggcaccgcgcccagtatgagccgctcttccacatgctcagtggccccgaggcctatgtgttcacctgcatcaaccagacagcggagcagcaagagctggaggacgagcaacggcgtctgtgtgacgtgcagcccttcctgcccgtcctgcgcctggtggcccgtgagggcgaccgcgtgaagaagctcatcaactcacagatcagcctcctcatcggcaaaggcctccacgagtttgactccttgtgcgacccagaagtgaacgactttcgcgccaagatgtgccaattctgcgaggaggcggccgcccgccggcagcagctgggctgggaggcctggctgcagtacagtttccccctgcagctggagccctcggctcaaacctgggggcctggtaccctgcggctcccgaaccgggcccttctggtcaacgttaagtttgagggcagcgaggagagcttcaccttccaggtgtccaccaaggacgtgccgctggcgctgatggcctgtgccctgcggaagaaggccacagtgttccggcagccgctggtggagcagccggaagactacacgctgcaggtgaacggcaggcatgagtacctgtatggcaactacccgctctgccagttccagtacatctgcagctgcctgcacagtgggttgacccctcacctgaccatggtccattcctcctccatcctcgccatgcgggatgagcagagcaaccctgccccccaggtccagaaaccgcgtgccaaaccacctcccattcctgcgaagaagccttcctctgtgtccctgtggtccctggagcagccgttccgcatcgagctcatccagggcagcaaagtgaacgccgacgagcggatgaagctggtggtgcaggccgggcttttccacggcaacgagatgctgtgcaagacggtgtccagctcggaggtgagcgtgtgctcggagcccgtgtggaagcagcggctggagttcgacatcaacatctgcgacctgccccgcatggcccgtctctgctttgcgctgtacgccgtgatcgagaaagccaagaaggctcgctccaccaagaagaagtccaagaaggcggactgccccattgcctgggccaacctcatgctgtttgactacaaggaccagcttaagaccggggaacgctgcctctacatgtggccctccgtcccagatgagaagggcgagctgctgaaccccacgggcactgtgcgcagtaaccccaacacggatagcgccgctgccctgctcatctgcctgcccgaggtggccccgcaccccgtgtactaccccgccctggagaagatcttggagctggggcgacacagcgagtgtgtgcatgtcaccgaggaggagcagctgcagctgcgggaaatcctggagcggcgggggtctggggagctgtatgagcacgagaaggacctggtgtggaagctgcggcatgaagtccaggagcacttcccggaggcgctagcccggctgctgctggtcaccaagtggaacaagcatgaggatgtggcccagatgctctacctgctgtgctcctggccggagctgcccgtcctgagcgccctggagctgctagacttcagcttccccgattgccacgtaggctccttcgccatcaagtcgctgcggaaactgacggacgatgagctgttccagtacctgctgcagctggtgcaggtgctcaagtacgagtcctacctggactgcgagctgaccaaattcctgctggaccgggccctggccaaccgcaagatcggccacttccttttctggcacctccgctccgagatgcacgtgccgtcggtggccctgcgcttcggcctcatcctggaggcctactgcaggggcagcacccaccacatgaaggtgctgatgaagcagggggaagcactgagcaaactgaaggccctgaatgacttcgtcaagctgagctctcagaagacccccaagccccagaccaaggagctgatgcacttgtgcatgcggcaggaggcctacctagaggccctctcccacctgcagtccccactcgaccccagcaccctgctggctgaagtctgcgtggagcagtgcaccttcatggactccaagatgaagcccctgtggatcatgtacagcaacgaggaggcaggcagcggcggcagcgtgggcatcatctttaagaacggggatgacctccggcaggacatgctgaccctgcagatgatccagctcatggacgtcctgtggaagcaggaggggctggacctgaggatgaccccctatggctgcctccccaccggggaccgcacaggcctcattgaggtggtactccgttcagacaccatcgccaacatccaactcaacaagagcaacatggcagccacagccgccttcaacaaggatgccctgctcaactggctgaagtccaagaacccgggggaggccctggatcgagccattgaggagttcaccctctcctgtgctggctattgtgtggccacatatgtgctgggcattggcgatcggcacagcgacaacatcatgatccgagagagtgggcagctgttccacattgattttggccactttctggggaatttcaagaccaagtttggaatcaaccgcgagcgtgtcccattcatcctcacctatgactttgtccatgtgattcagcaggggaagactaataatagtgagaaatttgaacggttccggggctactgtgaaagggcctacaccatcctgcggcgccacgggcttctcttcctccacctctttgccctgatgcgggcggcaggcctgcctgagctcagctgctccaaagacatccagtatctcaaggactccctggcactggggaaaacagaggaggaggcactgaagcacttccgagtgaagtttaacgaagccctccgtgagagctggaaaaccaaagtgaactggctggcccacaacgtgtccaaagacaacaggcagtagtggctcctcccagccctgggcccaagaggaggcggctgcgggtcgtggggaccaagcacattggtcctaaaggggctgaagagcctgaactgcacctaacgggaaagaaccgacatggctgccttttgtttacactggttatttatttatgacttgaaatagtttaaggagctaaacagccataaacggaaacgcctccttcattcagcggcggtgctgggccccccgaggctgcacctggctctcggctgaggattgtcaccccaagtcttccagctggtggatctgggcccagcaaagactgttctcctcccgagggaaccttcttcccaggcctcccgccagactgcctgggtcctggcgcctggcggtcacctggtgcctactgtccgacaggatgcctcgatcctcgtgcgacccaccctgtgtatcctccctagactgagttctggcagctccccgaggcagccggggtaccctctagattcagggatgcttgctctccacttttcaagtgggtcttgggtacgagaattccctcatctttctctactgtaaagtgattttgtttgcaggtaagaaaataatagatgactcaccacacctctacggctggggagatcaggcccagccccataaaggagaatctacgctggtcctcaggacgtgttaaagagatctgggcctcatgtagctcaccccggtcacgcatgaaggcaaaagcaggtcagaagcgaatactctgccattatctcaaaaatctttttttttttttttttgagatggggtcttcctctgttgcccaggctggagtgcagtggtgcaatcttggctcactgtaacctccgcctcccaggttcaagtgattcttcttgcctcagcctcctgagtagctgggattacaggtgtgcaccacccgtacccagctaatttttgtattttagtagagacgggggtttcaccatgttggctgggctggtctcgaactcctgacctcaggtgatccacccgcctgagcctcccaaagtgctgggattacaggcatgagccaccacgcccggcccactctgccattgtctaagccacctctgaaagcaggttttaacaaaaggatgaggccagaactcttccagaaccatcacctttgggaacctgctgtgagagtgctgaggtaccagaagtgtgagaacgagggggcgtgctgggatctttctctctgactatacttagtttgaaatggtgcaggcttagtcttaagcctccaaaggcctggatttgagcagctttagaaatgcaggttctagggcttctcccagccttcagaagccaactaactctgcagatggggctaggactgtgggcttttagcagcccacaggtgatcctaacatatcaggccatggactcaggacctgcccggtgatgctgttgatttctcaaaggtcttccaaaactcaacagagccagaagtagccgcccgctcagcggctcaggtgccagctctgttctgattcaccaggggtccgtcagtagtcattgccacccgcggggcacctccctggccacacgcctgttcccagcaagtgctgaaactcactagaccgtctgcctgtttcgaaatggggaaagccgtgcgtgcgcgttatttatttaagtgcgcctgtgtgcgcgggtgtgggagcacactttgcaaagccacagcgtttctggttttgggtgtacagtcttgtgtgcctggcgagaagaatattttctatttttttaagtcatttcatgtttctgtctggggaaggcaagttagttaagtatcactgatgtgggttgagaccagcactctgtgaaaccttgaaatgagaagtaaaggcagatgaaaagaaaaaaaaaaaaaaa